(19) United States
(12) Patent Application Publication
(10) Pub. No.: US 2026/0096749 A1
(43) Pub. Date: Apr. 9, 2026

(54) WEARABLE DEVICE AND METHOD FOR DETERMINING GLYCATED HEMOGLOBIN LEVEL

(71) Applicant: SAMSUNG ELECTRONICS CO., LTD., Suwon-si (KR)

(72) Inventors: Alexey Andreevich GAVRON, Moscow (RU); Elena Konstantinovna VOLKOVA, Saratov (RU); Vladislav Viktorovich SHIKHOV, Moscow (RU); Ekaterina Igorevna SEMERNYA, Moscow (RU); Anton Sergeevich LUKYANOV, Moscow (RU); Ariadna SCHUCK, Suwon-si (KR); Joongwoo AHN, Suwon-si (KR); Minho PARK, Suwon-si (KR); Younghyun KIM, Suwon-si (KR)

(73) Assignee: SAMSUNG ELECTRONICS CO., LTD., Suwon-si (KR)

(21) Appl. No.: 19/090,037

(22) Filed: Mar. 25, 2025

Related U.S. Application Data
(63) Continuation of application No. PCT/KR2025/003581, filed on Mar. 19, 2025.

(30) Foreign Application Priority Data
Oct. 8, 2024 (RU) 2024130316

(51) Int. Cl. A61B 5/145; A61B 5/00; A61B 5/1455

(52) U.S. Cl. CPC A61B 5/14532; A61B 5/0022; A61B 5/1455; A61B 5/681; A61B 5/7225; A61B 5/7267

(57) ABSTRACT

A wearable device for determining glycated hemoglobin level, includes at least three radiation sources; at least two radiation detectors configured to detect photoplethysmographic (PPG) signals of the at least three different wavelengths backscattered by the biological tissues; and a processor configured to: activate the radiation sources and the radiation detectors in pairs of measurement channels for the PPG signals; sequentially detect the PPG signals through each of the measurement channels; form, from the detected PPG signals, a dataset representing a time series of absorption of the PPG signals by the biological tissues of the user; generate a pre-processed dataset by carrying out a pre-processing of the dataset; and determine a glycated hemoglobin level based on the pre-processed dataset by a pre-trained machine learning model.

### CROSS-REFERENCE TO RELATED APPLICATIONS

This application is a continuation of International Application No. PCT/KR2025/003581, filed on Mar. 19, 2025, which is based on and claims priority to Russian Patent Application No. 2024130316, filed on Oct. 8, 2024, in the Russian Intellectual Property Office, the disclosures of which are incorporated by reference herein in their entireties.

### BACKGROUND

### 1. Field

The present disclosure relates to a wearable device for personally non-invasively, continuously and/or on-demand measuring a glycated hemoglobin level. In addition, the present disclosure relates to a method for determining a glycated hemoglobin level, the method being implemented in wearable devices such as modern smartwatches and fitness bracelets.

Embodiments can be implemented by using smartwatches, and also by specialized medical devices. Use of a multi-wavelength sensor and a signal pre-processing algorithm relates to particulars of one or more embodiments of the present disclosure.

### 2. Description of Related Art

For estimation of glycated hemoglobin (HbA1c), as well as total hemoglobin, it is necessary to measure an absolute concentration of glycated hemoglobin, but not a ratio between different fractions of hemoglobin. Long-term stability and fluctuations of the system become essential in that case, which must be taken into account by measurements at different wavelengths. Since glycated hemoglobin is just a relatively small part of total hemoglobin, it is necessary to develop an algorithm and device that is both sensitive to a concentration (level) of glycated hemoglobin and has the capacity to generalize (robustness).

An analysis performed by the World Health Organization (WHO) has shown that 529 million people are living with diabetes worldwide as of 2021, and that this number would increase up to almost 1.5 billion by 2050 (published in The Lancet on Jun. 22, 2023). Detection of abnormal concentrations of glycated hemoglobin (HbA1c) in human blood is the essential part of human health assessment. The glycated hemoglobin level is one of the early markers for diagnosing and monitoring of the diabetes mellitus. The HbA1c level indicates the average blood sugar content over a long period of time (up to three months), in contrast to a blood glucose level measurement, which gives an information on an instant blood glucose level.

Currently, many enzymatic and non-enzymatic electrochemical glucose sensors have been developed, however, such sensors are used in invasive methods.

Among possible technologies that allow HbA1c to be non-invasively measured, the visible, infrared (IR) and near IR spectroscopy is the most promising technique.

The main requirements to be considered when developing a HbA1c measurement system are:
designing a low-cost compact sensor applicable to wearable and portable devices;
non-invasive measurement;
high accuracy and repeatability of results; and
the ability to control health state and to adapt lifestyle.

At the same time, the main problems when measuring HbA1c are:
the difficulty of organizing of continuous real-time monitoring of HbA1c in a person due to inconvenience of a laboratory blood test and the need to involve medical personal;
the likelihood of incorrect measurement of a HbA1c level in patients with anemia or pregnant women; and
bulkiness and high cost of conventional general-purpose spectrometers.

Diabetes is a group of metabolic diseases in which a person has a high level of blood glucose either due to insufficient insulin production, or due to that the body's cells do not respond properly to insulin, or both. In order to be convinced in a sufficient insulin level, diabetics can check a blood sugar level several times a day, for example with a compact tester, without visiting hospitals. Constantly and timely monitoring of the glycated hemoglobin level makes it possible to prevent, promptly detect and control the progression of diabetes mellitus, as well as other disorders in a body function.

Hemoglobin is the main respiratory pigment and the main component of blood erythrocytes, performing important functions in the human body: transfer of inhaled oxygen from lungs to biological tissues and organs, and transfer of carbon dioxide from the tissues and organs to the lungs, where it is exhaled.

Glycated hemoglobin or glycohemoglobin (reference designation: hemoglobin HbA1c) is a biochemical indicator of blood that reflects an average content of blood sugar over a long time period and is not subjected to rapid changes, whereas, for example, a blood glucose level provides an idea of a blood sugar level only at the time of testing and quickly changes with food eating, physical activities, etc.

Glycated hemoglobin reflects the percentage of blood hemoglobin irreversibly bound to glucose molecules. Glycated hemoglobin is produced by the Maillard reaction between hemoglobin and glucose in blood, and this glycosylation reaction is irreversible. Increasing a blood glucose level in diabetes mellitus significantly accelerates this reaction, which results to increasing a blood glycated hemoglobin level. In other words, glycated hemoglobin is an integral indicator of glycemia over about two to three months. The higher the glycated hemoglobin level, the more often there was glycemia, that is, the elevated glucose level over the past three months and, accordingly, the greater the risk of developing complications of diabetes mellitus. Thus, the glycated hemoglobin level is currently a generally accepted indicator of the severity and degree of compensation for carbohydrate metabolism disorders.

Hemoglobin is a tetrameric protein, the molecule of which is formed by different types of polypeptide chains designated as α, β, γ, δ, ε, ξ. The composition of the molecule consists of 2 polypeptide chains of two different types, each of which binds one heme, therefore, there are four oxygen binding sites in the hemoglobin molecule. In the adult blood, the main hemoglobin is hemoglobin A (HbA), its proportion is 96-98% of the total amount of hemoglobin in the body, and its structure is described by the formula α2β2. The minor components are hemoglobin A2 (HbA2), a proportion thereof is about 2.5% of the total amount of hemoglobin and a structure of which is described by the formula α2δ2, and hemoglobin A3 (HbA3), the content of which is less than 1% of the total amount of hemoglobin. In addition, in the adult blood there is fetal hemoglobin F (HbF), which has a structure of α2γ2, but normally it is less than 1-1.5% of the total amount of hemoglobin in the adult blood. The hemoglobin molecule has an almost regular shape of a ball with a diameter of 55 Å, with the four chains arranged in the form of tetrahedron.

In arterial blood, substantially all (93-95%) hemoglobin HbA is bound to oxygen, i.e. it is in an oxygenated form, and serves to transfer oxygen. The rest of HbA (5-7%) is glycated hemoglobin and there are at least three options thereof: HbA1a, HbA1b, HbA1c, but only the HbA1c option, which is hemoglobin A modified by covalent addition of glucose thereto prevails quantitatively (4-6% of total hemoglobin) and provides a closer correlation with the severity of diabetes mellitus.

HbA1c contains one glucose molecule and is the main subtype of glycohemoglobin, constituting of 70-90% of the glycated fraction, the rest is HbA1a and HbA1b. HbA1c predominates in venous blood and is associated with a hyperglycemia level. The formation and disappearance of hemoglobin HbA1c is a continuous process, since every day new young erythrocytes replace the old dead ones in the blood channel. The degree of saturation of HbA with glucose and the rate of their binding with each other are directly dependent on the average content of it in the bloodstream over the past 90-120 days, wherein said period is due to the lifetime of hemoglobin-containing erythrocytes (red blood cells), which is about 120-125 days. Thus, a result of determining a glycated hemoglobin level reflects a glycemia level over a period of 60 to 120 days.

HbA1c values of 4% to 5.7% are considered to be normal, and values of between 5.7% and 6.4% signal a predisposition to diabetes. In diabetes, the HbA1c level is 6.5% or higher, which indicates a greater risk of development of retinopathy, nephropathy and other complications. The International Diabetes Federation recommends to maintain the HbA1c level below 6.5%, and the HbA1c value being above 8% means that diabetes is not well controlled and therapy should be altered.

Thus, the HbA1c level is proportional to the average blood glucose level over the past 6-12 weeks and constantly monitoring of this level is necessary to prevent the development of complications and to predict the development and phase of microvascular complications associated with diabetes.

Thus, the following target groups can be identified for which monitoring of a blood glucose/glycated hemoglobin level is necessary:
healthy people for the purpose of preventive control;
children with diabetes;
diabetic patients with abnormal renal glucose threshold;
patients with type I diabetes mellitus, insulin-dependent;
pregnant women with type II diabetes; and
other people when food ration or other habits are altered.

Hence, the glycated hemoglobin level is an important and significant indicator of human health and requires to be continuously monitored.

Invasive and non-invasive methods are used to determine a glycated hemoglobin level.

Invasive methods are laboratory techniques that include low-pressure ion exchange chromatography, capillary electrophoresis method, immunoturbidimetric method, high-performance liquid chromatography, colorimetric method. Blood for testing glycated hemoglobin can be sampled in a clinic treatment room, a biological material sampling point. Biomaterial samples are taken from a peripheral vein or a finger and delivered to a laboratory.

However, it should be noted that regular blood sampling may be painful and may create psychological load. In addition, since frequent blood samplings are able to cause a potential risk of infection to a person, frequent blood sugar testing is undesirable. With laboratory techniques, qualified personnel and expensive laboratory equipment are required for blood testing, and also waste should be disposed. Moreover, laboratory techniques are not suitable for continuously monitoring a glycated hemoglobin level, and patients cannot perform them self-acting. Therefore, non-invasive measurement methods based on the interaction of radiation with blood components have been widely developed.

The radiation used to determine a glycated hemoglobin level penetrates to different depths into a human biological tissue, depending on a wavelength, for example, the radiation of the green spectrum penetrates into the dermis, the red one-into the hypodermis, the near IR one-into the subcutaneous layer. In addition, all blood components such as plasma, erythrocytes, leukocytes, platelets, and human biological tissues such as sweat, hair bulbs, veins, arteries, capillary vessels, adipose tissue, epidermis, dermis, hypodermis and subcutaneous layer are absorbers, that is, blood components absorb and/or reflect radiation of different wavelengths to varying degrees. At the same time, in order to effective detect HbA1c, it is necessary that the applied radiation penetrates at least into the dermis and passes through blood vessels, veins, arteries, since HbA1c is a blood component, and therefore, acquisition of an optical signal from the blood is required to determine it.

Meanwhile, the main problem when measuring is reflection of radiation from the epidermis layer, in particular from the stratum corneum of the skin, which negatively affects a signal-to-noise ratio to impair the informativity of the acquired signal. Therefore, the signal quality expressed by the signal-to-noise ratio may not be sufficient to effective detect HbA1c. Hence, it is necessary to take into account or exclude the influence of noise.

In order to solve this problem, the present disclosure provides selective detection by the skin depth and takes into account the effect of radiation reflection from the upper skin layer.

In addition, in optical measurement methods in the visible and near-infrared radiation range, it should be taken into account that the absorption/reflection spectra of radiation by oxygenated, glycated and total hemoglobin overlap to each other, and therefore, they are difficult to be separated. Hence, separate labels are required for a type of hemoglobin, the spectrum of which is overlapped to HbA1c, and for HbA1c itself. At the same time, it is important not to lose the minimum difference between the absorption spectra of various components that overlap to each other.

In order to solve this problem, the present disclosure provides normalization of data by an “isobestic” point, while maximizing the differences in spectra for different absorbers, that is, the absorption spectra are normalized over a specific wavelength, for example, 850 nm, but not limited to.

Finally, as discussed earlier, glycated hemoglobin is an integral part of total hemoglobin and its content is only 4-6% of the total amount of hemoglobin in the blood, and therefore, a very high sensitivity of the device used to measure HbA1c is necessary.

The present disclosure takes into account the above-mentioned problems and provides a solution in which an innovative method of acquiring and measuring signals, special preparation and pre-processing of the acquired data are used, and which provides an accurate measurement of a glycated hemoglobin level in a non-invasive manner.

Nowadays, a number of devices for non-invasive measuring of glycated hemoglobin have been developed that reduce discomfort for patients. However, they are not always applicable for continuous monitoring. Moreover, there are no systems on the market, including wearable devices such as smartwatches or fitness bracelets intended to determine glycated hemoglobin in a non-invasive manner, with which a user is able to monitor HbA1c unassisted.

Thus, a problem of the technical field is the lack of a method and an easy-to-use device for measuring and continuously monitoring a blood parameter such as glycated hemoglobin, which take into account the influence of other blood components on a signal being measured. Meanwhile, the advantages of measuring of glycated hemoglobin with the device according to the present disclosure are rapid measurement, ease of use, and the possibility of long-term monitoring.

Hence, there is a demand for a device and method for non-invasively measuring a glycated hemoglobin level, which provide accurate measurement and are suitable for non-professional use. In other words, an easy-to-use device is required, for example, a wearable device for non-invasively, simply monitoring a glycated hemoglobin level. However, the principles of the present disclosure can be used not only in the wearable device. Possible products in which the method according to one or more embodiments of the present disclosure is used are wearable devices such as smartwatches or smart bracelets, stationary diagnostic tools, household appliances and gadgets for personal medical monitoring.

### SUMMARY

According to an aspect of the disclosure, a wearable device for determining a glycated hemoglobin level, includes: at least three radiation sources configured to irradiate biological tissues of a user with radiation of at least three different wavelengths; at least two radiation detectors, each of the at least two radiation detectors being configured to detect photoplethysmographic (PPG) signals representing the radiation of the at least three different wavelengths backscattered by the biological tissues; and at least one processor configured to: activate the at least three radiation sources and the at least two radiation detectors in pairs of measurement channels for the PPG signals; sequentially detect the PPG signals through each of the measurement channels by switching the measurement channels during a measurement cycle of the PPG signals; form, from the detected PPG signals, a dataset including a time series of values of the PPG signals relative to the at least three different wavelengths and relative to lengths of the measurement channels, and which defines an absorption of the PPG signals by the biological tissues of the user; generate a pre-processed dataset by carrying out a pre-processing of the dataset; and determine a glycated hemoglobin level based on the pre-processed dataset by a pre-trained machine learning model.

Each measurement channel includes a switching frequency, in a range of 25 to 100 Hz, and a measurement cycle of at least 60 seconds.

The pre-processing of the dataset may include filtering artifacts from the signals and frequency filtering.

The wearable device may further include: a housing; a battery; a memory; an input and output device; and a communication module accomodated in the housing, wherein the memory is configured to store a database with results of determining the glycated hemoglobin level, the input and output device is configured to input additional data and to display the glycated hemoglobin level, and the communication module is configured to communicate with any of a remote server and a cloud storage.

The at least three different wavelengths may be in a visible (VIS) to near-infrared (NIR) spectral range.

The at least three different wavelengths may be any of 525 nm, 575 nm, 630 nm, 660 nm, 730 nm, 805 nm, 850 nm, 940 nm, and 990 nm.

Two wavelengths of the at least three different wavelengths may be 525 nm and

575 nm.

The at least three radiation sources may include light-emitting diodes.

The at least two radiation detectors may include photodiodes.

The dataset may be an array of time series, each time series representing a plurality of sequential measurements, point by point, during the measurement cycle, and a single measurement in each of the measurement channels is represented by one of the following equations: A1=(εHbA1c(λ1)×cHBA1c+εtHb(λ1)×ctHb+εHHb(λ1)×cHHb+ . . . +εabsorber1(λ1)×cabsorber1+εabsorber2(λ1)×cabsorber2+ . . . )×d1, A2=(εHbA1c(λ1)×cHbA1c+εtHb(λ1)×ctHb+εHHb(λ1)×cHHb+ . . . +εabsorber1(λ1)×cabsorber1+εabsorber2(λ1)×cabsorber2+ . . . )×dN, . . . . AN=(εHbA1c(λn)×cHbA1c+εtHb(λn)×ctHb+εHHb(λn)×cHHb+ . . . +εabsorber1(λn)×cabsorber1+εabsorber2(λn)×cabsorber2+ . . . )×dN, where A1 . . . AN is a total absorption in the measurement channels 1 . . . . N, respectively, λ1 . . . λn is a wavelength of radiation, d1 . . . dN is a length of a measurement channel, ε is a molar absorption coefficient of a particular absorber, and c is a concentration of the particular absorber.

The equations may further include additional parameters representing at least one of an ambient temperature, a heart rate, and body movements.

The equations may be based on an isobestic point at a wavelength of 730 nm, 805 nm, or 850 nm.

The equations may further include parameters representing an influence of dark noise and individual characteristics of a body.

The machine learning model may be pre-trained by using a plurality of datasets acquired during a measurement cycle of at least 300 seconds and correlated with reference glycated hemoglobin level data measured by a laboratory technique.

The machine learning model may be trained by using different measurement channels, different radiation wavelengths, and reflects influence of additional parameters, dark noise, individual characteristics of a body, and an isobestic point.

The wearable device may be configured to be worn on a wrist.

The wearable device may be a smart device.

According to an aspect of the disclosure, a method for determining a glycated hemoglobin level, performed by a wearable device, includes: irradiating, by at least three radiation sources, biological tissues of a user with radiation of at least three different wavelengths, detecting, by at least two radiation detectors, photoplethysmogram (PPG) signals representing the radiation of the at least three different wavelengths backscattered by the biological tissues, wherein the detecting the PPG signals includes: forming measurement channels for the PPG signals by activating pairs of the at least three radiation sources and the at least two radiation detectors; sequentially detecting the PPG signals by switching the measurement channels during a measurement cycle; forming, from the detected PPG signals, a dataset which includes a time series of values of the PPG signals relative to the at least three different wavelengths and relative to lengths of the measurement channels, and which defines an absorption of the PPG signals by the biological tissues of the user; generating a pre-processed dataset by pre-processing the dataset; and determining a glycated hemoglobin level based on the pre-processed dataset by a pre-trained machine learning model.

Each measurement channel may include a switching frequency, in a range of 25 to 100 Hz, and a measurement cycle of at least 60 seconds.

The pre-processing of the dataset may include filtering artifacts from the signals and frequency filtering.

The at least three different wavelengths may be in a visible (VIS) to near-infrared (NIR) spectral range.

The at least three different wavelengths may be any of 525 nm, 575 nm, 630 nm, 660 nm, 730 nm, 805 nm, 850 nm, 940 nm, and 990 nm.

Two of the at least three different wavelengths may be 525 nm and 575 nm.

The at least three radiation sources may include light-emitting diodes.

The at least two radiation detectors may include photodiodes.

The dataset may be an array of time series, each time series representing a plurality of sequential measurements, point by point, during the measurement cycle, and a single measurement in each of the measurement channels may be represented by one of the following equations: A1=(εHbA1c(λ1)×cHbA1c+εtHb(λ1)×ctHb+εHHb(λ1)×cHHb+ . . . +εabsorber1(λ1)×cabsorber1+εabsorber2(λ1)×cabsorber2+ . . . )×d1, A2=(εHbA1c(λ1)×cHBA1c+εtHb(λ1)×ctHb+εHHb(λ1)×cHHb+ . . . +εabsorber1(λ1)×cabsorber1+εabsorber2(λ1)×cabsorber2+ . . . )×dN, . . . . AN=(εHbA1c(λn)×cHbA1c+εtHb(λn)×ctHb+εHHb(λn)×cHHb+.+εabsorber1(λn)×cabsorber1+εabsorber2(λn)×cabsorber2+ . . . )×dN, where A1 . . . AN is a total absorption in the measurement channels 1 . . . N, respectively, λ1 . . . λn is a wavelength of radiation, d1 . . . dN is a length of a measurement channel, ε is a molar absorption coefficient of a particular absorber, and c is a concentration of the particular absorber.

The equations may further include additional parameters representing any of an ambient temperature, a heart rate, and body movements and are based on an isobestic point at a wavelength of any of 730 nm, 805 nm, and 850 nm.

The machine learning model may be pre-trained using a plurality of datasets acquired during a measurement cycle of at least 300 seconds and correlated with reference glycated hemoglobin level data measured by a laboratory technique.

The machine learning model may be trained by using different measurement channels, different radiation wavelengths, and reflects influence of additional parameters, dark noise, individual characteristics of a body, and an isobestic point.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 illustrates first and second key particulars (KP1 and KP2) according to one or more embodiments of the present disclosure;

FIG. 2A schematically illustrates an exemplary positional relationship of radiation sources and radiation detectors at side of a back cover of a wearable device according to one or more embodiments;

FIG. 2B illustrates particulars of selecting a wavelength of radiation used according to one or more embodiments;

FIG. 2C illustrates particulars of selecting a separation distance between a radiation source and a radiation detector according to one or more embodiments;

FIG. 3A illustrates absorption spectra of various types of hemoglobin in a visible to near-IR radiation range according to one or more embodiments;

FIG. 3B schematically illustrates transmission spectra of whole blood according to one or more embodiments;

FIG. 3C schematically illustrates transmission spectra of whole blood according to one or more embodiments;

FIG. 3D schematically illustrates transmission spectra of whole blood according to one or more embodiments;

FIG. 4A illustrates in more detail a step of selecting the separation distance between the radiation source and the radiation detector according to the first key particular according to one or more embodiments of the present disclosure;

FIG. 4B illustrates particulars of recording of signals by a skin depth according to one or more embodiments;

FIG. 5 illustrates the influence of an optical path on multiple measurement channels according to one or more embodiments;

FIG. 6 illustrates the influence of a radiation wavelength on multiple measurement channels according to one or more embodiments;

FIG. 7 illustrates preparation of a dataset for further pre-processing according to KP2 of the present disclosure according to one or more embodiments;

FIG. 8 illustrates particulars of training a model for predicting a glycated hemoglobin level according to one or more embodiments;

FIG. 9A illustrates an exemplary layout of the wearable device and the front and rear view of the smartwatch according to according to one or more embodiments of the present disclosure;

FIG. 9B illustrates an exemplary layout of the wearable device and the front and rear view of the smartwatch according to according to one or more embodiments of the present disclosure

FIGS. 10A, 10B, 10C, 10D, 10E, 10F, and 10G illustrate exemplary embodiments of the present disclosure;

FIG. 11 illustrates occasional events in the daily life of a user of the wearable device according to according to one or more embodiments of the present disclosure and changes in this case in levels of various blood components;

FIG. 12A schematically illustrates options of exemplary displaying of HbA1c level measurement results within 24 hours and variances of HbA1c measurement results within several months on a screen of the wearable device according to the according to one or more embodiments of the disclosure in the S-Health app; and

FIG. 12B schematically illustrates a graph displayed on the screen of the wearable device according to one or more embodiments in the S-Health app and allowing the variance of a glycated hemoglobin level from a target value to be monitored.

### DETAILED DESCRIPTION

According to one or more embodiments, there is provided a wearable device for determining a glycated hemoglobin level in blood of a user, the wearable device comprising at least three radiation sources configured to irradiate the biological tissues of the user with radiation of at least three different wavelengths, at least two radiation detectors, each of the at least two radiation detectors being configured to detect photoplethysmographic (PPG) signals representing the radiation of the at least three different wavelengths backscattered by the biological tissues, and at least one processor configured to: activate the at least three radiation sources and the at least two radiation detectors in pairs of measurement channels for the PPG signals, sequentially detect the PPG signals through each of the measurement channels by switching the measurement channels during a measurement cycle of the PPG signals, form, from the detected PPG signals, a dataset including a time series of values of the PPG signals relative to the at least three different wavelengths and relative to lengths of the measurement channels, and which defines an absorption of the PPG signals by the biological tissues of the user, generate a pre-processed dataset by carrying out a pre-processing of the dataset; and determine a glycated hemoglobin level based on the pre-processed dataset by a pre-trained machine learning model.

According to one or more embodiments, each measurement channel comprises a switching frequency, in a range of 25 to 100 Hz, and a measurement cycle of at least 60 seconds.

According to one or more embodiments, the pre-processing of the dataset comprises filtering artifacts from the signals and frequency filtering.

According to one or more embodiments, the wearable device further comprises a housing, a battery, a memory, an input and output device, and a communication module accommodated in the housing, wherein the memory is configured to store a database with results of determining the glycated hemoglobin level, wherein the input and output device is configured to input additional data and to display the glycated hemoglobin level, and wherein the communication module is configured to communicate with any of a remote server and a cloud storage.

According to one or more embodiments, the at least three different wavelengths are in a visible (VIS) to near-infrared (NIR) spectral range.

According to one or more embodiments, the at least three different wavelengths are any of 525 nm, 575 nm, 630 nm, 660 nm, 730 nm, 805 nm, 850 nm, 940 nm, and 990 nm.

According to one or more embodiments, two wavelengths of the at least three different wavelengths are 525 nm and 575 nm.

According to one or more embodiments, the at least three radiation sources comprise light-emitting diodes.

According to one or more embodiments, the at least two radiation detectors comprise photodiodes.

According to one or more embodiments, the dataset is an array of time series, each time series representing a plurality of sequential measurements, point by point, during the measurement cycle, and wherein a single measurement in each of the measurement channels is represented by one of the following equations:

$$\begin{matrix}\begin{matrix}{A}_{1} = ( {ε}_{HbA 1 c} ( {λ}_{1} ) \times {c}_{HbA 1 c} + {ε}_{tHb} ( {λ}_{1} ) \times {c}_{tHb} + {ε}_{HHb} ( {λ}_{1} ) \times {c}_{HHb} + \\ … + {ε}_{absorber 1} ( {λ}_{1} ) \times {c}_{absorber 1} + {ε}_{absorber 2} ( {λ}_{1} ) \times {c}_{absorber 2} + … ) \times {d}_{1} ,\end{matrix} \\ \begin{matrix}{A}_{2} = ( {ε}_{HbA 1 c} ( {λ}_{1} ) \times {c}_{HbA 1 c} + {ε}_{tHb} ( {λ}_{1} ) \times {c}_{tHb} + {ε}_{HHb} ( {λ}_{1} ) \times {c}_{HHb} + \\ … + {ε}_{absorber 1} ( {λ}_{1} ) \times {c}_{absorber 1} + {ε}_{absorber 2} ( {λ}_{1} ) \times {c}_{absorber 2} + … ) \times {d}_{N} ,\end{matrix} \\ … \\ \begin{matrix}{A}_{N} = ( {ε}_{HbA 1 c} ( {λ}_{n} ) \times {c}_{HbA 1 c} + {ε}_{tHb} ( {λ}_{n} ) \times {c}_{tHb} + {ε}_{HHb} ( {λ}_{n} ) \times {c}_{HHb} + \\ … + {ε}_{absorber 1} ( {λ}_{n} ) \times {c}_{absorber 1} + {ε}_{absorber 2} ( {λ}_{n} ) \times {c}_{absorber 2} + … ) \times {d}_{N} ,\end{matrix}\end{matrix}$$

where A₁ . . . A_{N} is a total absorption in the measurement channels 1 . . . . N, respectively,
λ₁ . . . λₙ is a wavelength of radiation,
d₁ . . . d_{N} is a length of a measurement channel,
ε is a molar absorption coefficient of a particular absorber, and
c is a concentration of the particular absorber.

According to one or more embodiments, the equations further comprise additional parameters representing at least one of an ambient temperature, a heart rate, and body movements.

According to one or more embodiments, the equations are based on an isobestic point at a wavelength of 730 nm, 805 nm or 850 nm.

According to one or more embodiments, the equations further comprise parameters representing an influence of dark noise and individual characteristics of a body.

According to one or more embodiments, the machine learning model is pre-trained by using a plurality of datasets acquired during a measurement cycle of at least 300 seconds and correlated with reference glycated hemoglobin level data measured by a laboratory technique.

According to one or more embodiments, the machine learning model is trained by using different measurement channels, different radiation wavelengths, and reflects influence of additional parameters, dark noise, individual characteristics of a body, and an isobestic point.

According to one or more embodiments, the wearable device is configured to be worn on a wrist.

According to one or more embodiments, the wearable device is a smart device.

One or more embodiments of the disclosure also relate to a method for determining a glycated hemoglobin level in blood of a user, performed by a wearable device, the method comprising: irradiating, by at least of three radiation sources, biological tissues of the user with radiation of at least three different wavelengths, detecting, by at least two radiation detectors, photoplethysmographic (PPG) signals representing the radiation of the at least three different wavelengths backscattered by the biological tissues, wherein the detecting the PPG signals comprises: forming measurement channels for the PPG signals by activating pairs of the at least three radiation sources and the at least two radiation detectors, sequentially detecting the PPG signals by switching the measurement channels during a measurement cycle, forming, from the detected PPG signals, a dataset which includes time series of values of the PPG signals relative to the at least three different wavelengths and relative to lengths of the measurement channels and which defines an absorption of the PPG signals by the biological tissues of the user, generating a pre-processed dataset by pre-processing the dataset; and determining a glycated hemoglobin level based on the pre-processed dataset by a pre-trained machine learning model.

According to one or more embodiments of the method, each measurement channel comprises a switching frequency, in a range of 25 to 100 Hz, and a measurement cycle of at least 60 seconds.

According to one or more embodiments of the method, the pre-processing of the dataset comprises filtering artifacts from the signals and frequency filtering.

According to one or more embodiments of the method, the at least three different wavelengths are in a visible (VIS) to near-infrared (NIR) spectral range.

According to one or more embodiments of the method, the at least three different wavelengths are any of 525 nm, 575 nm, 630 nm, 660 nm, 730 nm, 805 nm, 850 nm, 940 nm and 990 nm.

According to one or more embodiments of the method, two of the at least three different wavelengths are 525 nm and 575 nm.

According to one or more embodiments of the method, the at least three radiation sources comprise light-emitting diodes.

According to one or more embodiments, the at least two radiation detectors comprise photodiodes.

According to one or more embodiments of the method, the dataset is an array of time series, each time series representing a plurality of sequential measurements, point by point, during the measurement cycle, and wherein a single measurement in each of the measurement channels is represented by one of the above-mentioned equations.

According to one or more embodiments of the method, the equations further comprise additional parameters representing any of an ambient temperature, a heart rate, and body movements and are based on an isobestic point at a wavelength of any of 730 nm, 805 nm or 850 nm.

According to one or more embodiments of the method, machine learning model is pre-trained using a plurality of datasets acquired during a measurement cycle of at least 300 seconds and correlated with reference glycated hemoglobin level data measured by a laboratory technique.

According to one or more embodiments of the method, the machine learning model is trained by using different measurement channels, different radiation wavelengths, and reflects influence of additional parameters, dark noise, individual characteristics of a body, and an isobestic point.

One or more embodiments of the disclosure also relate to a system for determining a glycated hemoglobin level in blood of a user, the system comprising the wearable device according to the above-mentioned embodiments and a remote server and/or a cloud storage, wherein the system comprises means of communication between the wearable device and the remote server and/or the cloud storage.

According to one or more embodiments of the system, a database including user profile data and the results of previous determining a glycated hemoglobin level is stored on a storage device, the remote server and/or the cloud storage, wherein the system further comprises means for accessing the user profile data and the results of previous determining a glycated hemoglobin level from external devices via the remote server and/or the cloud storage.

One or more embodiments of the wearable device, method and system allow for non-invasively, accurately self-measuring a glycated hemoglobin (εHbA1c) level and are suitable for non-professional use.

Hereinafter, particulars of the present disclosure and exemplary embodiments of the disclosure will be described. Persons skilled in the art will be understood that various exemplary embodiments should not be construed as limiting the scope of the present disclosure, and that other tangible and technical means equivalent or similar to those listed below may be used by skilled persons to perform various operations, functions, method steps, etc., described below. The present detailed description is not intended to limit the scope of the present disclosure, which is defined only by the appended claims.

It should be noted that the principles of the present disclosure are given with respect to measuring a glycated hemoglobin level. However, persons skilled in the art will be understood that the present disclosure is not limited to and can be used to determine other user blood parameters, for example, other types of hemoglobin by adapting hardware and also by training and using an appropriate prediction model.

Note that, as used herein, the terms “radiation” and “light,” “radiation source” and “LED”; “radiation detector,” “photodetector” and “photodiode”; “absorber” and “chromophore”; “measurement separation,” “measurement channel” and “optical path”; “prediction model” and “machine learning model”; “weight” and “weight factor” can be used interchangeably.

Hereinafter, key particulars of the present disclosure will be described with reference to the appended drawings.

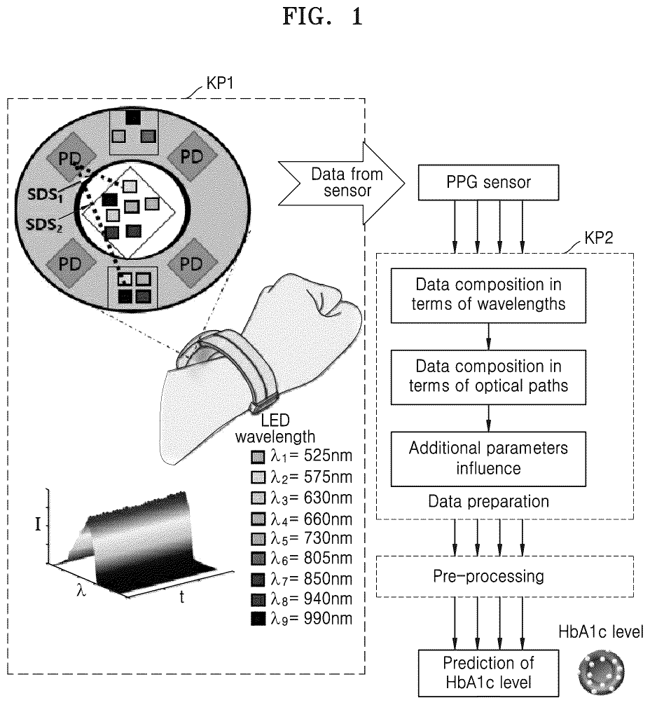

FIG. 1 illustrates first and second key particulars of one or more embodiments of the present disclosure.

As an example, FIG. 1 shows the back side of a wearable device such as a smartwatch, at which several LEDs and several photodetectors (PD), used to emit radiation and to detect the emission after being passed through the tissues of a user of the device, are located. As shown in FIG. 1, four photodetectors are located along the periphery of the back side of the wearable device through every 90°, four LEDs are at a “6 o'clock” position, three LEDs are at “12 o'clock” position (the “peripheral” group of LEDs) and seven LEDs are in the center of said back side (the “central” group of LEDs). However, the layout of LEDs and photodetectors is not limited to that shown in FIG. 1, and many other layouts including a plurality of LEDs and photodetectors, that provide different combinations of optical paths, can be implemented. At the same time, the wearable device according to the present disclosure should comprise at least three light-emitting diodes configured to emit radiation of at least three different wavelengths in the range of 500-1000 nm (or fewer LEDs if they are capable of emitting radiation of several different wavelengths), and at least two photodetectors for detecting radiation of said wavelengths. The selection of said number of the radiation sources and detectors is caused by the following considerations:
at least two photodetectors allow signals to be measured at different distances or optical paths that the radiation of each of the wavelengths travels from the LED to the photodetector; and
at least three wavelengths allow for selecting radiation for maximum response of HbA1c and other types of hemoglobin in the range used.

Eventually, there is an objective to separate the absorption spectrum of the absorber of interest, namely glycated hemoglobin, from other spectra.

As an example, said at least three radiation wavelengths used may be selected from the following: 525 nm, 575 nm, 630 nm, 660 nm, 730 nm, 805 nm, 850 nm, 940 nm, and 990 nm, but not limited to. Note that the wavelength values may differ by +/−5% from the above and the exact values will depend on the selection of appropriate LEDs. In a preferred embodiment, radiation of at least 525 nm and/or 575 nm wavelengths is used, since the greatest absorption (response) of HbA1c is observed at these wavelengths. Other wavelengths are auxiliary and are necessary to take into account the influence of other components as much as possible. In particular, HbO₂ has the greatest response at 630 nm and 660 nm wavelengths, tHb and RHb—at 850 nm, 940 nm and 990 nm, and 730 nm, 805 nm and/or 850 nm are used to normalize and obtain an isobestic point or points.

The presence and positional relationship of a plurality of radiation sources and radiation detectors at different distances from each other allow signals to be detected at different wavelengths and at different distances by activating each of all possible LED/photodetector pairs. For example, as shown in the lower part of FIG. 1, when one LED/photodetector pair is activated, the LED/photodetector separation distance is short (SDS₁), when another LED/photodetector pair is activated, the separation distance is long (SDS₂). From the point of view of the distance between the radiation source and the radiation detector, a greater distance provides a greater optical path that radiation photons travel, and therefore their greater interaction with biological tissues. At the same time, in the context of one or more embodiments of the present disclosure, short and long separation distances are referred so as to indicate merely a variety of distances at which signals are detected, so specific optical path lengths are not specified. Since there is a network of capillaries in both the dermis and the epidermis, and the presence of a PPG signal is a necessary and sufficient condition for using a phase in an algorithm, the introduction of concepts and the assignment of specific parameters to them is not technically important according to one or more embodiments. Nevertheless, as an example, it is noted that the separation distances between the radiation sources and detectors on the back surface of existing wearable devices are from 0.5 to 10 mm. Since the depth of the epidermis layer is 0.07-1.4 mm and the depth of the dermis layer is 0.3-3.0 mm depending on a body area, gender, body constitution, only as an example, but not limitation, it is noted that an optical path of about 0.5-7.0 mm is implied under the short measurement channel, and an optical path of about 7.0-15.0 mm, preferably 7.0-10.0 mm is implied under the long channel. It should be noted that in some different pairs, a LED and a photodetector are located at almost the same separation distances, which allow a signal to be measured in channels of the same length, but at different radiation wavelengths.

Thus, the layout of the sensors according to one or more embodiments allows for detecting signals of both different wavelengths and at different distances between LED/photodetector.

Thus, the first key particular (KP1) of one or more embodiments of the disclosure, consisting in matching a source/detector pair and a wavelength to further determine a glycated hemoglobin level, is provided by:
the presence of multiple radiation sources capable of emitting radiation of different wavelengths;
the presence of multiple radiation detectors capable of detecting the radiation of different wavelengths that has passed through the biological tissues of the user; and
acquiring signals from all radiation sources almost simultaneously, point by point.

Due to implementation of KP1, one or more embodiments of the disclosure provides acquiring of time series relative to different radiation wavelengths and relative to different optical paths, respectively, from which a dataset used for further processing is formed.

The second key particular (KP2) of one or more embodiments of the disclosure, consisting in the data preparation (decomposition of multi-signal components) and analysis, is provided by:
taking into account the influence of using of different optical paths (data preparation);
taking into account the influence of using of different wavelengths (data preparation); and
taking into account the influence of additional parameters to render the solution more robust, i.e. greater generalization and accuracy.

Thus, the steps of the method for determining a glycated hemoglobin level generally are:
1. Acquisition of data by means of PPG sensor(s) containing LEDs and photodetectors.
2. Preparation of the acquired data which includes a data composition in terms of wavelengths and a data composition in terms of optical paths.
3. Preliminary processing (pre-processing) of the prepared data.
4. Prediction of a glycated hemoglobin level by means of a pre-trained machine learning model.

Use of algorithms to extract an information is implied here under machine learning. Machine learning in one or more embodiments of the disclosure is performed on the basis of publicly known principles and techniques disclosed, in particular in a publication by Christopher M. Bishop, Pattern Recognition and Machine Leaning, Springer, 2006/1/.

One or more embodiments of the disclosure provides the following advantageous effects:
1. Reliable estimation of a glycated hemoglobin level.
2. Real-time measurement and estimation.
3. Robust and accurate solution.
4. Ability to define the prediabetic and diabetic level.
5. Using FDA requirements free approach to measure a glycated hemoglobin level.

Further, the solution according to one or more embodiments by the present inventors will be disclosed in detail with references to figures.

The problem of other solutions is mainly in insufficient sensitivity of the method for determining a glycated hemoglobin level. It should be noted that the reflection of radiation from the upper skin layers and from the skin surface, i.e. the stratum corneum, results to deterioration in a signal-to-noise ratio. In addition, the HbA1c content is only 4-6% of the total amount of hemoglobin in the blood, and therefore, the device used to measure HbA1c should have a very high sensitivity.

In order to solve this problem, it is necessary to pursue that the signals of diffuse reflection spectra include sufficient absorption information from HbA1c, and it is also necessary to eliminate the problems of reducing the measurement accuracy when a lot of variables (other types of hemoglobin or other absorbers with interference with HbA1c) would exist.

Hereinafter, KP1 and KP2 of one or more embodiments of the present disclosure will be discussed in more detail with reference to figures. KP1 consists in matching radiation sources and radiation detectors for measuring a glycated hemoglobin level.

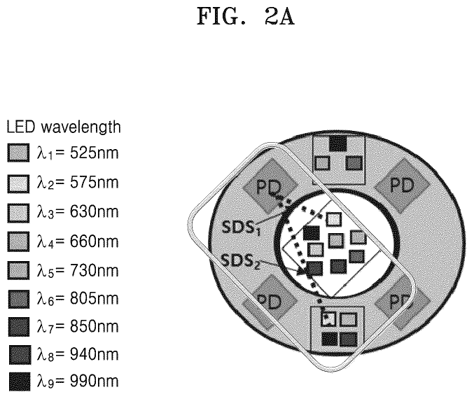

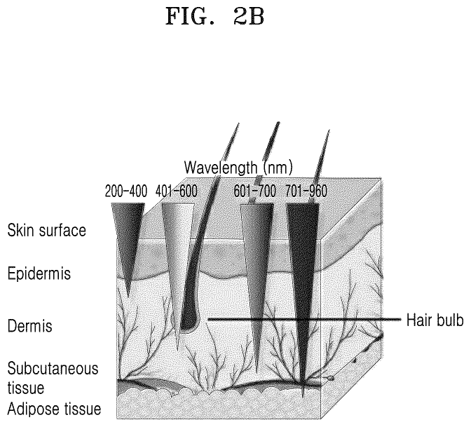

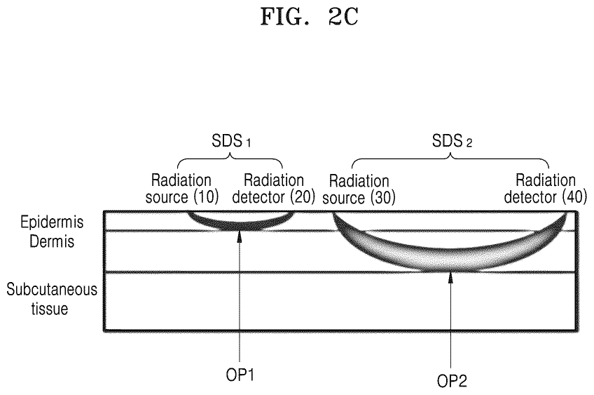

FIG. 2A schematically illustrates an exemplary positional relationship of radiation sources and radiation detectors at side of a back cover of a wearable device. The radiation sources can be, for example, LEDs, and the radiation detectors can be, for example, photodetectors (PD). The layout of LEDs and photodetectors may include several groups of LEDs and several photodetectors. As an example, but not limited to, FIG. 2A shows four photodetectors being located along the periphery of the back side of a wearable device every 90°, and three groups of LEDs being located at a “6 o'clock,” “12 o'clock” position (“peripheral” LEDs) and in the central part (“central” LEDs) of the back side of the device. When a HbA1c level is measured by the wearable device, different LED/photodetector pairs are alternately (one-by-one) activated, that is, first, a signal is measured by means of the first pair, then by means of the second pair and alternately till the last pair activated, then the measurement order is repeated. The measurements are performed during a cycle of at least 60 seconds. It should be noted that, as will be described below, a specific set of LED/photodetector pairs is activated, based on which a prediction model is trained. However, one or more embodiments of the disclosure are not limited to the specific set of LED/photodetector pairs, and any other set can be used, provided that appropriate training of the prediction model is performed, as will be described below. As shown in FIG. 2A as an example, a pair of one of the central LEDs emitting radiation, for example, of a wavelength of 575 nm, and one of the photodetectors can be activated first. This pair provides recording a signal with a radiation wavelength of 575 nm at a short distance LED/photodetector (SDS₁). Then a pair of one of the peripheral LEDs emitting radiation of the same wavelength of 575 nm and the same photodetector can be activated. This pair provides recording a signal with a radiation wavelength of 575 nm at a long distance LED/photodetector (SDS₂). A plurality of other LED/photodetector pairs are then sequentially activated to acquire a set of signals with a source wavelength selection and with a LED/photodetector separation distance selection. Each pair of LED/photodetector is a channel for measuring a signal of a predetermined wavelength at a predetermined distance defined by the configuration of the device according to the present disclosure. The switching (sampling) frequency for each measurement channel may be, for example, 25 Hz, 30 Hz, 40 Hz, 60 Hz, 70 Hz, 80 Hz, 90 Hz, 100 Hz or more, and preferably is 50 Hz, i.e. for each LED/photodetector pair, 50 measurements per second are performed, but not limited to. Accordingly, if there are, for example, six different LED/photodetector pairs, then 6 pairs×50 Hz=300 measurements per second, and 300 meas./s×60 s=18000 measurements per cycle are performed. It is obvious that with increasing of frequency, an amount of detected data, and therefore, the accuracy of measurements increases. However, the measurement frequency is limited by the device hardware and battery capabilities. In particular, if the operating frequency is too high, the battery will be quickly discharged.

FIG. 2B illustrates particulars of selecting a wavelength of radiation used. As known, radiation of different wavelengths, but of the same power, penetrates at different distances into a human biological tissue. In addition, radiation of different wavelengths is differently absorbed by different absorbers. Hence, it is necessary to properly select wavelengths of the radiation used to acquire distinct signals for HbA1c and other biological tissues, as well as other types of hemoglobin with interference for HbA1c measurement. FIG. 2B shows the penetration depths of radiation of different wavelengths of the same power into human biological tissues. For example, radiation of a wavelength of 200-400 nm penetrates into the epidermis layer and can penetrate into the upper part of the dermis layer, i.e. provides acquiring a weak signal from blood vessels, and therefore, it is not suitable for determining of glycated hemoglobin, radiation of a wavelength of 401-600 nm penetrates approximately to the middle of the dermis layer, radiation of a wavelength of 601-700 nm penetrates into almost the entire depth of the dermis layer, radiation of a wavelength of 701-960 nm penetrates into the subcutaneous layer and adipose tissue. This is the reason to select the radiation sources for one or more embodiments of the disclosure which are capable of emitting radiation with a wavelength in the range of 500-1000 nm. Meanwhile, it should be noted that the radiation power of the LEDs is adjusted so that the radiation of any wavelength used would reach the blood vessels to acquire signals useful for the HbA1c determination.

FIG. 2C illustrates particulars of selecting a separation distance between a radiation source and a radiation detector (Source-Detector Separation, SDS). An optical path along which the radiation passes from the source through the biological tissues of a user of the device to the detector depends on the distance between the radiation source and the radiation detector. By increasing the radiation power, the optical path can be increased, but in general, the greater the separation distance, the greater the optical path. Selecting a proper SDS to obtain a satisfactory HbA1c measurement accuracy is performed as follows:
1) The short channel (e.g. SDS₁) signal passing through the epidermis layer and partially the dermis layer is used to determine a HbA1c level and to measure the interference “noise” signal. A signal that passes at a short distance between the LED (e.g. 10) and the photodetector (e.g. 20) and reaches the epidermis layer and only partially the dermis layer is implied under the short channel signal. The distance which radiation travels within biological tissues depends on the radiation wavelength and power.
2) The long channel (e.g. SDS₂) signal passing mainly through the dermis layer is used to determine a HbA1c level. A signal passing at such a distance between the LED (e.g. 30) and the photodetector (e.g. 40) that exceeds the travel distance of the short channel signal, and reaching the dermis layer is implied under the long channel signal.

The length of each of said channels depends on the separation distance between the radiation source and the radiation detector and is the optical path that radiation photons travel between the radiation source and the radiation detector. For example, a length of a first optical path OP1 that radiation photons travel between the radiation source 10 and the radiation detector 20 depends on the separation distance SDS₁ between the radiation source 10 and the radiation detector 20. A length of a second optical path OP2 that radiation photons travel between the radiation source 30 and the radiation detector 40 depends on the separation distance SDS₂ between the radiation source 30 and the radiation detector 40. Therefore, from this point of view, the distance between the radiation source and the radiation detector is directly related to the optical path length. At the same time, when the distance between the radiation source and the radiation detector increases, the optical path length usually increases.

It should be noted that biological tissues are relatively transparent to the radiation used in one or more embodiments of the disclosure, which makes non-invasively measuring at a depth of a few millimeters being safe for human. In addition, as shown in FIG. 2C, a travel trajectory of radiation photons through the tissues resembles a parabola, a shape of which is sometimes described by the term “banana-shape.” Therefore, due to changing of the positional relationship of the LEDs and the photodetectors and using of the appropriate radiation wavelengths, it is possible to accurately define an area of interest, that is, the area for gathering signals from the required skin layers by depth. More specifically, the hardware of the device according to one or more embodiments of the disclosure are configured so that a PPG signal (pulse wave) is observed at each wavelength and each LED/photodetector pair, i.e. at each of the possible optical paths. This means that the radiation used reaches the blood vessels, i.e. arteries, veins, arterioles, venules, capillaries, that is, it reaches the “substance” in which HbA1c is “contained.” In other words, each LED is configured such (including by adjusting a current strength) that the radiation emitted by it reaches the blood vessels and then the reflected and scattered radiation is recorded by a photodetector, regardless of the distance therebetween. That is, all the activated LED/photodetector pairs provide the acquisition of signals from the skin layers that include the blood vessels. Thus, all signals contain a useful information regarding a glycated hemoglobin level.

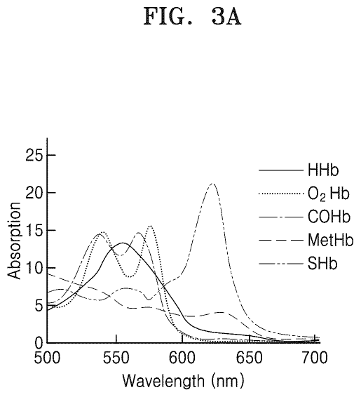

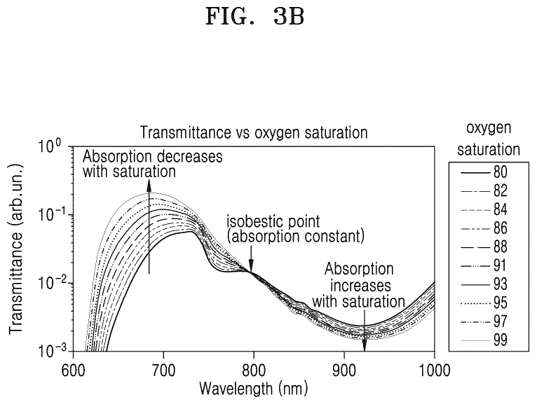

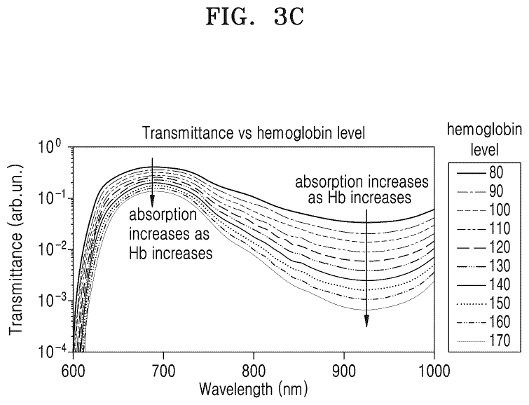

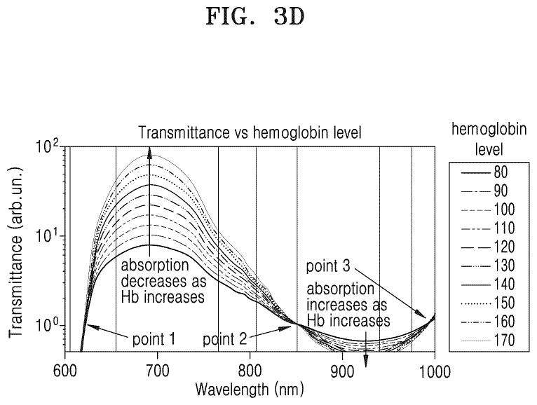

FIG. 3A illustrates absorption spectra of various types of hemoglobin in the visible to near-IR radiation range. In particular, as an example, absorption spectra of deoxyhemoglobin (HHb), oxyhemoglobin (HbO₂), carboxyhemoglobin (HbCO), methemoglobin (MetHb) and sulfhemoglobin (SHb) are shown. Hemoglobin and all its derivatives are colored proteins that absorb radiation differently in the visible to near-infrared spectral range and thus have characteristic absorption spectra. Therefore, it is necessary to obtain separate labels (characteristic information) for both HbA1c and the Hb type that interferes with the HbA1c determination. At the same time, a machine learning model for predicting a glycated hemoglobin level should have multi-output prediction that takes into account, in addition to glycated hemoglobin, at least oxygenated hemoglobin and total hemoglobin. As known, different radiation wavelengths are usually used to detect different types of hemoglobin: 525 nm and 575 nm for HbA1c, 630 nm and 660 nm for HbO₂, 850 nm, 940 nm, 990 nm for tHb and RHb, and wavelengths of 730 nm, 805 nm and 850 nm are suitable for to be used as an “isobestic” point. For example, a spectrum (spectra) may be normalized by one or more wavelengths. For example, when the spectrum is normalized by 805 nm and 850 nm wavelengths, two differently pre-processed spectra are obtained that can be further used.

FIG. 3B, FIG. 3C and FIG. 3D schematically illustrate transmission spectra of whole blood. Graphs of FIG. 3B, FIG. 3C and FIG. 3D show the difference between relative and absolute measurements of optical properties of whole blood: dependence of optical density on an oxygen saturation rate (graph of FIG. 3B); dependence of optical density on a hemoglobin concentration (graph of FIG. 3C); dependence of optical density on a hemoglobin concentration after being normalized by 850 nm (graph of FIG. 3D).

More particularly, the graph of FIG. 3B shows the transmittance of whole blood as a function of the radiation wavelength at different blood oxygen saturation (oxygenation), that is, at different content of oxygenated hemoglobin. Particulars of the illustrated spectra are decrease in absorption with increasing of oxygenation in the range of about 620 nm to less than 800 nm and increase in absorption with increasing of oxygenation in the range of about more than 800 nm to 1000 nm. Meanwhile, when the radiation wavelength is about 800 nm, there is an isobestic point in which absorption of whole blood is substantially the same at different oxygenation. The graph of FIG. 3C shows the transmittance of whole blood as a function of the radiation wavelength at different total hemoglobin concentrations. Particulars of the illustrated spectra are increase in absorption with increasing a hemoglobin concentration within the entire radiation range shown from about 600 nm to 1000 nm. Meanwhile, at different wavelengths, in particular in the ranges of 600-800 nm and 800-1000 nm, absorption increases to a varying degree with increasing a hemoglobin concentration. Therefore, the transmission spectra illustrated in the graph of FIG. 3C may be normalized, for example, at a wavelength of 850 nm, at which the difference in absorption at different hemoglobin concentrations becomes significant. The graph of FIG. 3D illustrates such normalized transmission spectra, particulars of which are decrease in absorption with increasing a hemoglobin concentration in the range from about 620 nm to less than 850 nm and increase in absorption with increasing a hemoglobin concentration in the range from about 850 nm to less than 990 nm. Note that after the spectra are normalized, there are three isobestic points, i.e., 620 nm, 850 nm and 990 nm.

Thus, the absorption of radiation by other tissue components can be detected by the deviation of the normalized transmission at one of the isobestic points from unity. These considerations have enabled the present inventors to radically simplify the signal pre-processing and feature extraction procedure for a machine learning (ML) model.

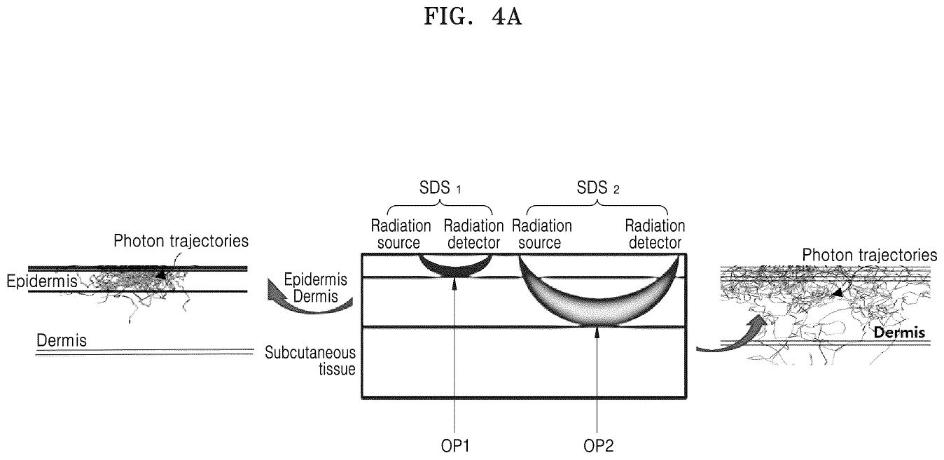

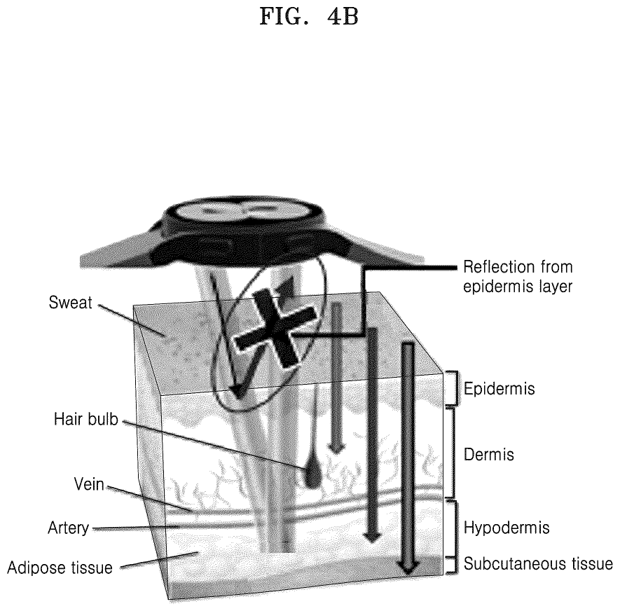

FIG. 4A illustrates in more detail a step of selecting the separation distance between the radiation source and the radiation detector according to the first key particular of one or more embodiments of the present disclosure. As discussed hereinabove, in one or more embodiments of the disclosure, different radiation sources and radiation detectors are matched to obtain different optical paths therebetween. In other words, the possibility of detecting of radiation is provided for all source/detector pairs to be activated. By this matching, the signal is provided to be recorded at short (SDS₁) and long (SDS₂) source/detector separation distances, which are also referred herein to as short channels and long channels, respectively. Note that the separation distances mentioned as short and long ones are not limited to and can have an intermediate length.

FIG. 4A schematically shows an exemplary diagram of propagation paths of radiation photon through skin tissues. An experiment performed according to Monte-Carlo simulations is given from an article by Murad M. Althobaiti, Ibraheem A₁-Naib, Optimization of Dual-Channel Near-Infrared Non-Invasive Glucose Level Measurement Sensors Based On Monte-Carlo Simulations, IEEE Photonics Journal, Vol. 13, No. 3, June 2021, (https://www.researchgate.net/publication/351596942)/2/. As known, the skin consists of three main layers: epidermis which is the outermost skin layer, dermis and hypodermis, and the layer of subcutaneous tissue is located deeper. The size of the considered model is a 16×16×16 mm³ cubic volume with a voxel size of 0.1 mm. The radiation sources and detectors were simulated as pencil sources (with a narrow beam) and disk photodetectors, respectively. For each simulation run, a photon fluence distribution was acquired for each source and detector channel. An ideal short SDS₁ channel should have no sensitivity to the dermis because the photon of the short SDS₁ channel is generally trajected from radiation source to radiation detector through a first optical path OP1, and an ideal long SDS₂ channel should exhibit the highest sensitivity distribution from the dermal tissue because the photon of the long SDS₂ channel is generally trajected from radiation source to radiation detector through a second optical path OP2. Obtaining this ideal scenario of the short SDS₁ channel is impractical because the defined geometry and the sensitivity distributions are continuous functions. The practical aim is to minimize dermis sensitivity (DS) at a reasonable signal-to-noise ratio (SNR) for optimal short channel separation and to maximize DS for optimal long channel separation. For each model, the DS was computed which describes how a particular channel is sensitive to the dermis layer contents comparative to the overall sensitivity (see below equation (eq.) 1):

$$D S = 100 \times \frac{{\sum }_{Dermis} PMDF}{{\sum }_{total} PMDF} , \qquad Eq . ( 1 )$$

where DS is a voxel-wise sum (processing of one voxel in one pass) of the photon measurement density function (PMDF) in the dermis layer divided by the sum of the whole PMDF for each model.

In other words, FIG. 4A shows a diagram of a dual-channel near-IR sensor for monitoring a blood glucose level. Two sources and two detectors are arranged at different distances from each other (SDS). When SDS₁ is short, photons pass mainly through the epidermis layer, and only a small part thereof reaches the dermis layer, that is, the radiation interacts with the surface layers in which capillaries are located. When SDS₂ is long, photons pass mainly through the dermis layer, to a lesser extent through the epidermis layer, and a small part thereof can reach the subcutaneous layer, that is, the radiation interacts with the deeper layers in which blood vessels such as capillaries, veins and arteries are located. It should be noted that the main contribution to noise is defined by the reflection of radiation from the skin stratum corneum, which acts as a “screen” for radiation.

The selection of a proper source/detector separation distance (SDS) to obtain a satisfactory HbA1c measurement accuracy should be ideally made taking into account the following:
the short channel (SDS₁) signal originating from the epidermis layer and partially the dermis layer is used to measure the interference “noise” signal; and
the long channel (SDS₂) signal originating from the dermis layer and containing useful information about the HbA1c level is used to measure HbA1c.

Meanwhile, the short channel signal can be removed from the long channel signal. Thus, noise can be removed, which leads to simplification of subsequent signal processing. However, in real conditions, it is difficult to implement such a configuration of the device. Therefore, in one or more embodiments of the disclosure, all measurement channels are associated with blood flow in different blood vessels, as discussed earlier, so both the short channel signals and the long channel signals contain an information about HbA1c.

FIG. 4B illustrates particulars of recording of signals by a skin depth. The elimination of the influence of the short channel signal on the measurement result provides the effect of one or more embodiments of the present disclosure, consisting in the selectivity of signal detection by a skin depth and in taking into account the influence of radiation reflection from the upper skin layers for the purpose of high sensitive detection of HbA1c.

FIG. 4B schematically illustrates the human skin layers, namely the epidermis, dermis, hypodermis and subcutaneous layer (subcutaneous tissue), as well as a wet film (sweat), hair bulbs, veins, arteries, capillaries, adipose tissue located in these layers. In addition, the depth to which green, red and near-IR radiation of the same power penetrates into a human skin is schematically illustrated.

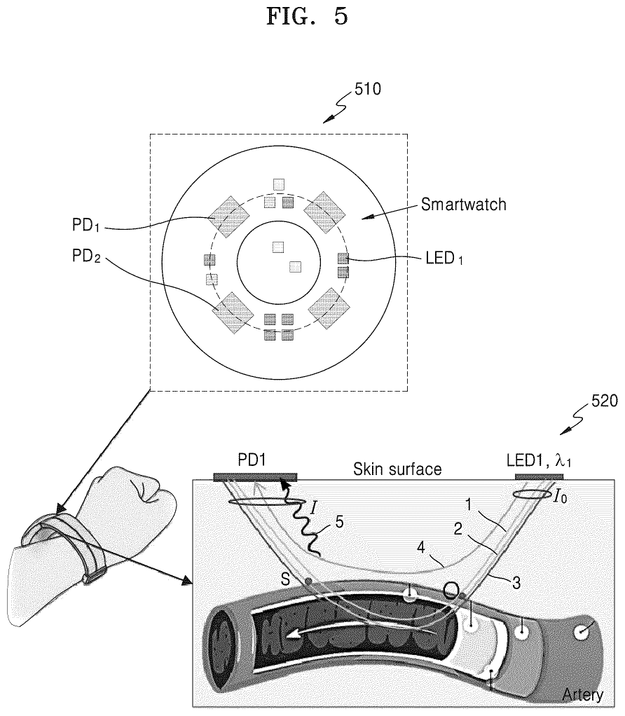

FIG. 5 illustrates optical path influence to multiple measurement channels to explain KP2 of one or more embodiments of the present disclosure, consisting in simultaneous multi-signal components decomposition (data preparation) and analysis. In the upper part 510 of FIG. 5, a smartwatch view from the side of a back cover is schematically shown. Four photodetectors are located along circumference at the back side of the smartwatch at intervals of 90° and thirteen LEDs are located here, however, their number can vary. For example, two LEDs are at a “3 o'clock” position, four LEDs are at a “6 o'clock” position, two LEDs are at a “9 o'clock” position, three LEDs are at “12 o'clock” position (the “peripheral” group of LEDs) and two LEDs are in the center of said back side (the “central” group of LEDs). LEDs are capable of emitting radiation of at least three wavelengths selected from 525 nm, 575 nm, 630 nm, 660 nm, 730 nm, 805 nm, 850 nm, 940 nm, and 990 nm.

As an example, the total absorption by human tissues of radiation of a wavelength λ emitted by one radiation source LED₁ and detected by two radiation detectors PD₁ and PD₂ being located at different distances from it, is determined by the following equations:

$${A}_{1} = ( {ε}_{HbA 1 c} ( {λ}_{1} ) \times {c}_{HbA 1 c} + {ε}_{tHb} ( {λ}_{1} ) \times {c}_{tHb} + {ε}_{HHb} ( {λ}_{1} ) \times {c}_{HHb} + … + {ε}_{absorber 1} ( {λ}_{1} ) \times {c}_{absorber 1} + {ε}_{absorber 2} ( {λ}_{1} ) \times {c}_{absorber 2} + … ) \times {d}_{1} , \qquad Eq . ( 2 )$$

$${A}_{2} = ( {ε}_{HbA 1 c} ( {λ}_{1} ) \times {c}_{HbA 1 c} + {ε}_{tHb} ( {λ}_{1} ) \times {c}_{tHb} + {ε}_{HHb} ( {λ}_{1} ) \times {c}_{HHb} + … + {ε}_{absorber 1} ( {λ}_{1} ) \times {c}_{absorber 1} + {ε}_{absorber 2} ( {λ}_{1} ) \times {c}_{absorber 2} + … ) \times {d}_{2} , \qquad Eq . ( 3 )$$

where A is a total absorption of radiation in a particular channel at a specific measurement time, ε is a molar absorption coefficient of an absorber, mol/l,
c is a concentration of various absorbers of the radiation, that is, radiation-attenuating species, which are skin, muscle, adipose and connective tissues, bones, hair, different blood components, such as different types of hemoglobin, different chromophores, etc., mol⁻¹×cm⁻¹, wherein chromophores include, for example, nucleic acids, proteins, lipids, provitamin B3, porphyrin, and
d is a length of an optical path along which the radiation passes, cm.

Thus, one or more embodiments of the disclosure take into account all the absorbers with which the radiation interacts in its propagation path. It is obvious that the optical path lengths d₁ and d₂ are different for users of the device due to different physiology, for example, skin thickness and color, amount of adipose tissue, etc. However, as will be understood by a person skilled in the art, this does not affect the HbA1c determination accuracy in one or more embodiments of the disclosure.

Thus, both equations contain the same unknown values of the concentrations of different chromophores, including an unknown value of the concentration of the target component, i.e., glycated hemoglobin, and differ only by the optical path length. Meanwhile, each point measurement at a specific radiation wavelength can be expressed by one of the above-mentioned equations. Hence, in the above example of the device layout with six different LED/photodetector pairs, each pair is defined by its own equation of total absorption, that is, there are six equations. Meanwhile, at a measurement frequency of 50 Hz and a measurement cycle duration of 1 minute, there are 3,000 measurements for each channel, or, in other words, 3,000 solutions to the corresponding equation at specific measurement points of time. Accordingly, for the six channels, there are 18,000 measurements or solutions to the equations at specific measurement points of time.

The lower part 520 of FIG. 5 schematically shows a LED and a photodetector located in contact with the skin of a user of the device, and an exemplary optical path that radiation of a wavelength of A passes through the user's tissues. Points O and S approximately limit a part of the optical path that the radiation of a wavelength of 2 passes through the user's blood vessels, such as arteries, veins, arterioles, venules and capillaries, in which absorption is defined by the walls of the vessels and the various blood components, in particular the absorption depends inter alia on a level of the target component, i.e., glycated hemoglobin. Thus, the initial intensity (power) I₀ of radiation of a wavelength of λ₁ emitted by the LED₁ is attenuated to the intensity I of the radiation detected by the photodetector PD1 due to the absorption by various blood components, such as plasma water, mineral salts, formed elements, proteins, including various types of hemoglobin, and other biological tissues. As an example, FIG. 5 shows the radiation attenuation due to the HbA1c absorption, A_{HbA1c}(λ₁, t₀) (indicated by numeral 1 in figure), the total Hb absorption, A_{tHb}(λ₁, t₀) (2), the oxygenated Hb absorption, A_{HbO2} (λ₁, t₀) (3) at the optical path d(λ₁, t₀) (e.g. optical path from point O to point S), and the absorption by biological tissues (4, e.g., adipose and muscle tissues, bones, skin, water, etc.) at the optical path d′(λ₁, t₀). In addition, the intensity I is also affected by the intensity of dark noise (5). It is also obvious that radiation of different wavelengths differently interact with blood components and biological tissues (adipose, muscle, bone), so the absorption of radiation of a wavelength λ_{N} will have its own particulars and differ from the absorption at λ₁.

As a result of measuring the absorption at the same wavelength in different measurement channels, the effect of one or more embodiments of the present disclosure is provided, consisting in that different optical paths allow different total absorptions to be calculated, i.e., different information necessary to predict HbA1c to be obtained.

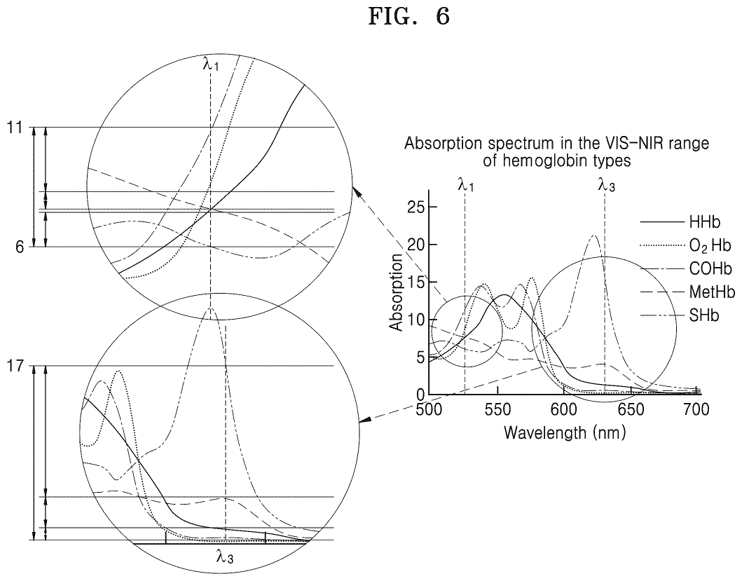

The influence of lengths of the measurement channels on acquiring of signals at the same wavelength was discussed above. Hereinafter, the influence of the radiation wavelength on multiple measurement channels will be considered with reference to FIG. 6.

In the right part of FIG. 6, as an example, graphs of absorption of various types of hemoglobin, i.e., deoxyhemoglobin (HHb), oxyhemoglobin (HbO₂), carboxyhemoglobin (HbCO), methemoglobin (MetHb), sulfhemoglobin (SHb) depending on the wavelength in the range of 500-700 nm are shown. It can be seen from the graphs that different types of hemoglobin differently absorb radiation depending on the wavelength. In the left part of FIG. 6, portions of the absorption graphs of various types of hemoglobin at wavelengths λ₁ (about 525 nm) and λ₃ (about 630 nm) are illustrated in an enlarged view. It can be seen from the graphs that at the wavelength λ₁, the absorption by each of these types of hemoglobin is from 6 to 11%, whereas at the wavelength λ₃, the main contribution to absorption is made by SHb—about 17%, and the absorption by other types of hemoglobin is rather weak. As an example, the radiation absorption only by certain types of hemoglobin was illustrated in FIG. 6, but other absorbers present in the blood and biological tissues interact with radiation in a similar manner. Hence, there are obvious differences in the radiation absorption by different absorbers at different wavelengths.

Thus, the total absorption of radiation by human tissues and blood in the measurement channels having approximately the same length d₁ at different wavelengths λ₁ and λ₃ is defined by the following equations:

$${A}_{1} = ( {ε}_{HbA 1 c} ( {λ}_{1} ) \times {c}_{HbA 1 c} + {ε}_{tHb} ( {λ}_{1} ) \times {c}_{tHb} + {ε}_{HHb} ( {λ}_{1} ) \times {c}_{HHb} + … + {ε}_{absorber 1} ( {λ}_{1} ) \times {c}_{absorber 1} + {ε}_{absorber 2} ( {λ}_{1} ) \times {c}_{absorber 2} + … ) \times {d}_{1} , \qquad Eq . ( 4 )$$

$${A}_{2} = ( {ε}_{HbA 1 c} ( {λ}_{3} ) \times {c}_{HbA 1 c} + {ε}_{tHb} ( {λ}_{3} ) \times {c}_{tHb} + {ε}_{HHb} ( {λ}_{3} ) \times {c}_{HHb} + … + {ε}_{absorber 1} ( {λ}_{3} ) \times {c}_{absorber 1} + {ε}_{absorber 2} ( {λ}_{3} ) \times {c}_{absorber 2} + … ) \times {d}_{2} , \qquad Eq . ( 5 )$$

Thus, both equations contain the same unknown values of concentrations of different blood components, including an unknown value of the concentration of the target component, i.e., glycated hemoglobin, and differ in that the data are acquired at different radiation wavelengths, which are known and defined by the LEDs used. Meanwhile, each point measurement in a specific measurement channel can be expressed by one of the above-mentioned equations.

As a result of measuring of absorption in the measurement channels having approximately the same length at different wavelengths, the advantage is provided that consists in that the use of radiation of different wavelengths allows even more different total absorptions to be calculated, that is, more different information necessary to predict HbA1c to be obtained. The more information is obtained, the more accurately the glycated hemoglobin level can be predicted. An amount of information obtained is limited only by the hardware capabilities.

According to KP2 of one or more embodiments of the present disclosure, the influence of additional parameters on the multiple measurement channels can also be taken into account, in particular as follows:
1) Use of an additional multiplier vector a in the equations:

$${A}_{1} = [ a \times ( {ε}_{HbA 1 c} ( {λ}_{1} ) \times {c}_{HbA 1 c} + … ) ] \times {d}_{1} + b . \qquad Eq . 6$$

The vector multiplier takes into account the influence of external and internal factors on the measurement results. For example, when an ambient temperature or motion artifacts increase, a signal amplitude increases, wherein it increases for the entire set of equations. Due to the use of the additional multiplier vector, additional stability of the system of equations and its solution are provided when changing internal/external conditions: a temperature, heartbeat, motion artifacts, etc.

Thus, a more robust and generalized solution is created, which takes into account the influence of internal and external factors.
2) Addition of an isobestic point, for example, 850 nm (λ₇):

$${A}_{5} = [ a \times ( {ε}_{HbA 1 c} ( {λ}_{7} ) \times {c}_{HbA 1 c} + … ) ] \times {d}_{5} + b . \qquad Eq . 7$$

After normalizing the equations by any wavelength, for example, by the wavelength λ₇, the equation term ε_{HbA1c}(λ₇)×c_{HbA1c} becomes zero. Thus, firstly, the system of equations is simplified, and, secondly, the exclusion of even one equation term allows differences between all absorbers for all the given equations to be maximized and a glycated hemoglobin level to be more accurately predicted.
3) Separation of total hemoglobin (tHb) as an absorber into the equation term separate from HbA1c (despite that HbA1c is 4% of tHb, i.e. it is a constituent part thereof), since total Hb and glycated Hb have different absorption properties when interacting with radiation of different wavelengths:

$${A}_{2} = [ a \times ( {ε}_{HbA 1 c} ( {λ}_{1} ) \times {c}_{HbA 1 c} + {ε}_{tHb} ( {λ}_{1} ) \times {c}_{tHb} + … ) ] \times {d}_{2} + b . \qquad Eq . ( 8 )$$

This allows an information on glycated hemoglobin and total hemoglobin to be separated for better, more accurate and stable prediction.
4) Use of an additional term vector b:

$${A}_{15} = [ a \times ( {ε}_{HbA 1 c} ( {λ}_{7} ) \times {c}_{HbA 1 c} + … ) ] \times {d}_{15} + b . \qquad Eq . ( 9 )$$

The additional term vector allows additional noise caused by individual particulars of a body, dark noise, etc. is taken into account and the solution to be made being more robust.

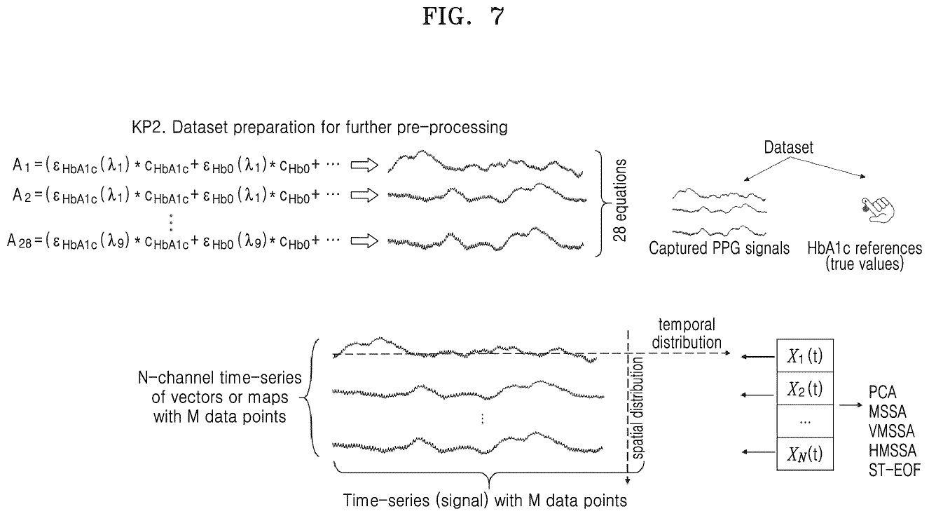

Hereinafter, preparation of a dataset for further pre-processing will be described with reference to FIG. 7. As an example, it is shown that the measurements were performed in 28 measurement channels, and accordingly, for each channel the total absorption is determined by the corresponding equation. In this case, the wearable device is provided with nine different LEDs and four photodetectors. The LEDs emitting the most important wavelengths for analysis, for example, 525 and 575 nm, form pairs with each photodetector, that is, each of the LEDs emitting radiation of the wavelength of 525 and 575 nm forms four measurement channels with the photodetectors, and the LEDs of other radiation wavelengths, for example, 660 nm may form 1 to 4 pairs with the photodetectors, for example, two pairs. The above exemplary embodiment is preferable taking into account the hardware of a modern Samsung smartwatch, but does not limit the claimed solution.

A point measurement of the pulse wave value (PPG signal) acquired by means of the device at a specific point of time is a solution to one of the above equations, which characterize values of total absorption of radiation after passing through biological tissues of a user and interacting with all possible absorbers in an optical path. As discussed earlier, the switching frequency for each measurement channel is preferably 50 Hz. Thus, for each of the 28 measurement channels, 50 measurement results per second, i.e. solutions to the corresponding equation, are obtained. In other words, the signal measured in each channel (PPG signal) consists of 50 sequential points per second, that is, 50 solutions to the corresponding equation per second are obtained in each measurement channel. A combination of the solutions to the equations is a time series of values acquired either relative to a predetermined wavelength or relative to a predetermined length of the measurement channel. Accordingly, the result, for example, of a 5-minute measurement will consist of 300 s×50=15,000 points for each measurement channel (each LED/photodetector pair activated). In an exemplary embodiment, there are 28 LED/photodetector pairs (combinations). Therefore, taking into account the measurement frequency in each channel being 50 Hz, switching between all 28 channels occurs at a frequency of 28×50 Hz=1400 Hz. Thus, in 1 second 1,400 solutions to the equations characterizing the total absorption in all channels are obtained.

It should be noted that at least a five-minute measurement cycle is required to acquire a dataset for training a machine learning model for prediction. When operating the wearable device in a glycated hemoglobin measurement mode, at least one-minute measurement cycle is required. It is expected that with further improvement of the machine learning model, the measurement time will decrease.

A photodetector is a device that converts optical energy into electrical one, so the photodetector measures the photocurrent or photosignal voltage (voltage shift) caused by the radiation entering it. Two photoeffects are used in photodetectors: photovoltaic and photoconductivity. The following demands are imposed to photodetectors of optical devices:
high sensitivity;
required spectral characteristics and broadband;
a low noise level;
operation speed;
long service life; and
the possibility to be used in integrated circuits together with optical amplifiers.

To a large extent, these requirements are met by photodiodes, i.e., devices operating on the basis of the photovoltaic effect, which are preferably used in the present disclosure.

Note again that in order to implement the present disclosure, at least three different radiation wavelengths are required, for example, three LEDs and two photodetectors disposed at different locations, to obtain at least six measurement channels, that is, six series of equations. In this case, there are 28 time series of the PPG signals for each user, acquired within one minute, which differ in the length of the measurement channels and/or the wavelength.

The next step is a pre-processing of the acquired dataset, which consists in properly arranging of signals and filtering. There may be artifacts and/or unnecessary certain frequencies in the signals that may interfere with proper prediction. In particular, it is necessary to remove the lowest frequency usually representing a trend of an acquired signal, since the trend is not related to the concentration of absorbers, but rather is related, for example, to heating of a hand from the wearable device over time. Filtering a signal allows such artifacts and/or frequencies to be detected and removed. But this should be done simultaneously for all signals in order to maintain the measured relationships between different wavelengths and measurement channels.

Carrying out a processing of signals in the present disclosure is based on publicly known principles and techniques disclosed, in particular, in a publication of Alan V. Oppenheim, Ronald W. Schafer, Digital Signal Processing, Prentice-Hall, 1975/3/.

According to the principles of one or more embodiments of the present disclosure, ratios between the acquired signals are important for accurate determining a glycated hemoglobin level, but not their absolute values. The combination of signals measured in each of the N measurement channels is a time series with M data points. The combination of all time series constitutes a dataset or a data array to be further processed. Meanwhile, it is important to process the time series simultaneously. Pretreatment can be performed by publicly known methods: Principal Component Analysis (PCA), Multivariate Singular Spectrum Analysis (MSSA), MSSA: vertical form (VMSSA), MSSA: horizontal form (HMSSA), Spatio Temporal Empirical Orthogonal Function (ST-EOF).

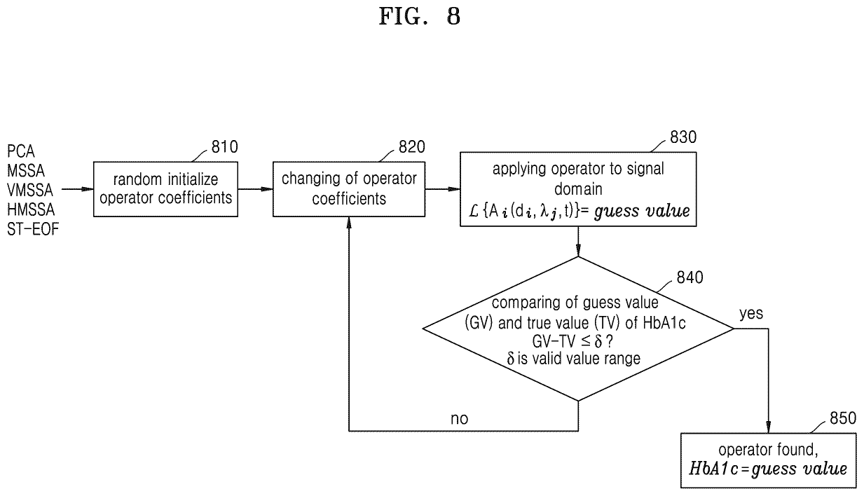

Training a machine learning model for predicting a HbA1c level (prediction model) will be described with reference to FIG. 8. A prediction model is a parametric function, and the task of its training is to select the parameters of the model so that it is best to describe the training data. In other words, the aim of optimization of the prediction model is to find operator of transformation from the signal domain to HbA1c values domain, which provides the reduction of prediction error δ, i.e., it is necessary to form a matrix of weight coefficients and train the matrix such that the weight coefficients provide the robustness of the prediction model.

In order to train the prediction model, it is necessary to form a so-called “dataset” or a training set. “Dataset” is acquired and prepared data, that is, a processed and structured data array. In fact, “dataset” is a plurality of the datasets described above, further including true or reference values of HbA1c measured by a laboratory technique and correlated with the corresponding datasets. A machine learning process is a complex data processing operation that involves sequentially entering an input data (dataset) into a model and comparing an output data with its true value, after which the weight coefficients are adjusted to reduce the error of the output data. This operation is performed repeatedly using data from the “dataset.” In the process of training and controlling the training quality, well-known methods of gradient descent and backpropagation of error are widely used. The gradient descent method allows for finding a local minimum of a function (which is a function of error) of several variables (weights or weight coefficients of input data) by successively small changing of these variables. The error backpropagation method allows for performing a procedure for correcting the weights in the direction from an output to inputs by using partial derivatives.

The training quality can be controlled by analyzing the errors of the machine learning model at its output when compared to the true value corresponding to a particular dataset. In the process of learning, as a rule, these errors decrease exponentially, but there may come a point of time when the error begins to grow. From this point of time, the so-called overtraining effect is fixed, when the learning process is advisable to be stopped.

The prediction model is trained on results of measurements collected in a group of 200 people to provide robustness of the algorithm used in machine learning of the prediction model, that is, good generalization, which allows the model to work reliably for any person. A plurality of datasets correlated with reference values of HbA1c is used to teach the prediction model. This trained model is used in the wearable device. Once being trained, the prediction model is capable for non-invasively predicting a glycated hemoglobin level while using a set of the measurement data acquired for each user of the wearable device as input data.

Meanwhile, in machine learning of the prediction model, the dataset, i.e. a combination of a set of data of the PPG signals acquired in 28 measurement channels, is correlated with a reference or true value of a HbA1c level measured using a known laboratory technique. Thus, the “dataset” is acquired, that includes the true value of glycated hemoglobin and an N-channel time series of vectors or maps with M data points and a time series with M data points. In other words, the dataset is acquired, that includes the signals relative a time, associated with different measurement channels and different wavelengths, i.e., a set of equations X₁(t), X₂(t) . . . X_{N}(t) obtained for a plurality of combinations of phases. Here, a measurement phase, i.e., measuring a single signal is understood under a phase or step, wherein supply of a current to a LED, emission, reception and processing of the single signal are performed in each phase or at each step. Thus, in each specific LED/photodiode channel, i.e. at the predetermined separation distance and wavelength, the combination of phases, for example, 50 phases per second, is obtained. Meanwhile, activating of different LED/photodiode pairs occurs according to a program that controls the operation of the wearable device in the HbA1c level measurement mode, that is, the program controls the execution of a plurality of phases one after another. In other words, X_{N}(t) includes a plurality of single measurements represented in the form of the equation for total absorption and performed during the measurement cycle t in the channel N at the specific point of time. The above set of equations is used as an input data when the prediction model is operated.

Meanwhile, after the pre-processing of the dataset:
810—random coefficients of an initialize operator (initial coefficients) are assigned;
820—the coefficients of the operator are changed;
830—the operator is applied to a signal domain, and a guess value of a glycated hemoglobin level is obtained: L{Aᵢ(dᵢ, λi, t}=guess value;
840—the guess value and the true value of the HbA1c level is compared;
850—if the difference between the guess value and the true value does not exceed the predetermined error δ (acceptable deviation range), it is concluded that the operator is found and the guess value corresponds to the hemoglobin level of the device user (850); otherwise, return to step 820 is performed.

In this case, a matrix of weight coefficient is kept in mind under the operator.

Thus, the prediction model is the matrix of weight coefficients W found when any neural network architecture is trained by means of “dataset,” that is, the trained parameters of the model. The matrix W is “sewn” into the wearable device, after which a user measures his/her data (PPG signals) Xᵢ by the wearable device, which, in a primitive version, is matrix-multiplied by the matrix of weight coefficients W and the prediction model directly provides a result of HbA1c estimation. Thus, the result of glycated hemoglobin estimation is obtained as:

$$S = {\sum }_{i = 1}^{N} Xi \times Wi , \qquad Eq . ( 10 )$$

where Xᵢ is a combination of phases, i.e. sets of PPG signals measured over a certain time duration in N measurement channels (input data for the prediction model), and
Wᵢ is a weight coefficient of the matrix for each measurement channel.

The use of the prediction model described above and a set of measurement results determined from the PPG signals allows for accurately determining a glycated hemoglobin level, taking into account the influence of other blood components.

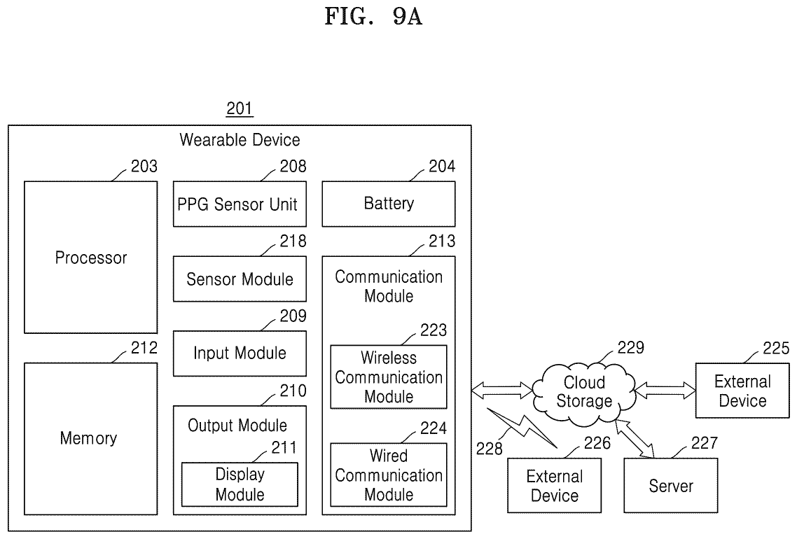

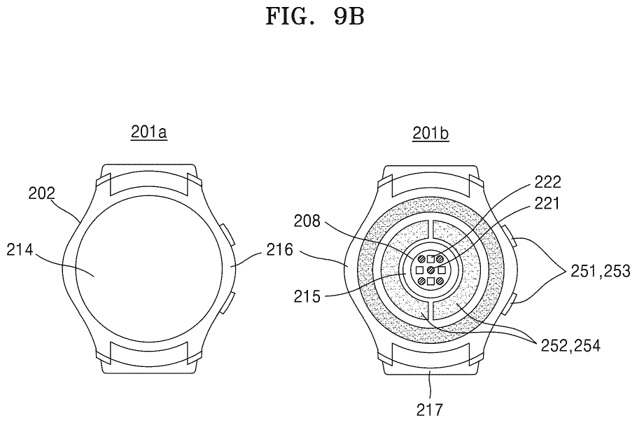

A wearable device 201 with function of determining of glycated hemoglobin is schematically illustrated with reference to FIGS. 9A and 9B.

FIG. 9A schematically illustrates a block diagram of a preferred embodiment of the wearable device 201 comprising a housing, a processor (for example, microprocessor) 203, a battery 204, a PPG sensor unit 208 including LEDs and photodetectors, an input module 209, an output module 210, a display module 211 including a display, a memory 212 including a volatile memory and a non-volatile memory, a communication module 213 including a wireless communication module 223 and a wired communication module 224, and a sensor module 218. The input module 209 and the output module 210 may be configured as a single input and output module. The sensor module 218 of the wearable device 201 may comprise sensors such as a bio-impedance (BIA) sensor, inertial movement sensors, gyroscopes, accelerometers (acceleration sensors), an electrocardiogram (EKG) measuring sensor, an atmospheric pressure sensor, a humidity sensor, a temperature sensor, etc. In embodiments, the wearable device 201 may be a smart device, in particular a smartwatch or a fitness bracelet, a medical wearable health monitoring device with function of determining of glycated hemoglobin, etc. The wearable device is an electronic wearable device and can be configured to establish a wired or wireless communication channel with external devices, for example, devices 225, 226 such as smartphones, wearable fitness bracelets, voice assistants, smart TVs, smartwatches, headphones, and so on, or with a server 227, and can be configured to transmit data via a network 228 or a cloud storage 229. In some embodiments, at least one of the components may be omitted from the electronic device 201, or one or more other components may be added to the electronic device 201.

The processor 203 can execute software to control at least one other component (for example, the sensor module 218) of the electronic device 201 and can perform various data processing or computations. As at least a part of data processing or computation, the processor 203 can load an instruction or data received from another component (for example, the sensor module 218, or the communication module 213) into the volatile memory, process the instruction or data stored in the volatile memory, and store the resulting data in the non-volatile memory. The processor 203 may include a main processing unit (for example, a central processing unit (CPU), an application processor (AP)), an auxiliary processor, etc., wherein the auxiliary processor may operate independently of or together with the main processing unit. The auxiliary processor may be implemented separately from or as a part of the main processing unit.

The processor 203 of the wearable device 201 is configured to execute a program that controls the operation of the device when determining a glycated hemoglobin level. In particular, the processor 203 controls the switching of measurement channels, processes the acquired information, and transmits HbA1c values determined on the basis of readings of the PPG sensor unit 208 to the output module 210 to inform a user. The processor 203 is configured to process the acquired signals, including filtering the PPG signals from motion artifacts, separating the PPG signals into AC and DC components of time series, extracting a set of features of the PPG signals.

The processor 203 is configured to load data received by means of the communication module 213 into the memory 212 and/or load data from the memory 212 into the communication module 213 for transmitting them to an external device, for example, the devices 225, 226, the server 227 or the cloud storage 229).

According to one or more embodiments, the main processing unit or auxiliary processor (for example, a neural network data processing device) may include a hardware structure designed to process an artificial intelligence model. An artificial intelligence model can be created by means of machine learning. Such leaning may be performed, for example, in the electronic device 201 itself on which the artificial intelligence model is operating, or may be performed via a separate server (for example, the server 227). Learning algorithms may include, but are not limited to, for example, supervised learning, unsupervised learning, partially supervised learning, or reinforcement learning. The artificial intelligence model can include many layers of an artificial neural network. An artificial neural network can be any of Attention-based Transformers, Deep Neural Network (DNN), Convolutional Neural Network (CNN), Recurrent Neural Network (RNN), Restricted Boltzmann Machine (RBM), Deep Belief Network (DBN), Bidirectional Recurrent DNN (BRDNN), Deep Q Network, or a combination of two or more of the aforementioned networks, but is not limited to the examples mentioned above. In addition to the hardware structure, the artificial intelligence model may further or alternatively include a software structure.

The memory 212 may store various data used by at least one component (for example, the processor 203, or the sensor module 218) of the electronic device 201. Various data may include, for example, software, and input data or output data for the associated command. The software may include, for example, an operating system (OS), middleware and/or applications.

The memory 212 in a preferred embodiment may be configured to store:
databases with results of measuring of glycated hemoglobin;
a date of collecting of each set of measurement data;
a time of collecting of each set of measurement data; and
a user profile data, etc.

The wearable device 201 with the PPG sensor unit 208 is capable of detecting, in a reflection mode, the radiation back-scattered and/or back-reflected from the biological tissues of the user, including skin, bones, blood, blood vessels. Detecting and measuring the back-scattered and/or back-reflected radiation are performed by means of photodetectors being at least one of a photodetector, a photoresistor, a phototransistor, a charge coupled device (CCD), or a complementary metal-oxide-semiconductor (CMOS) based device, or another photosensitive element, and a photocurrent-to-voltage converter.

In a preferred embodiment, the photodetector is a wide-band photodetector configured to detect and amplify a response of the radiation back-scattered and/or back-reflected from blood and tissues, which is a signal from a radiation source.

The wearable devices with a PPG sensor use a reflection mode to conveniently place the device, for example, on a user's hand. The PPG sensors being worn on the user's hand or other body part are in contact with the skin.

An electrical circuit of the PPG sensor may contain an amplifier, a high-pass filter (about 0.1 Hz) to cut off the constant component and obtain pulsating changes of a signal, a low-pass filter (about 30 Hz) to eliminate high-frequency noise, and also a microprocessor. The used frequency range depends on the circuit design. The PPG sensor may have a wireless module for transmitting data to an external device.

The bioimpedance measurement module (BIA sensor) may include a biocontact circuit (a user body contact circuit) comprising 4 electrodes, a module for measuring impedance comprising a current-voltage measurement module, a current-voltage circuit configuration module, an impedance calculation module, at least one leakage current compensation module, and optionally a microprocessor.

The battery 204 is configured to supply power to at least one component of the wearable device 201. The battery may be a primary cell or a secondary (rechargeable) cell and may be built into the wearable device or replaceable.

In a preferred embodiment of one or more embodiments of the present disclosure illustrated in FIG. 9B, the wearable device 201 is configured in the form of a smartwatch which is adapted to be placed on a user's wrist. In other embodiments of one or more embodiments of the present disclosure, the wearable device 201 may be configured to be placed on other parts of the user's body, for example, on a finger of hand.

FIG. 9B schematically illustrates the wearable device 201 in a top view 201a and a bottom view 201b. According to this embodiment, the housing 202 of the wearable device 201 includes a first side 214 (or a front surface), a second side (or a back surface) 215 and a side surface 216 surrounding a space between the first side 214 and the second side 215, and attachment elements 217 designed for detachably attaching the wearable device 201 to a wrist, for example, by means of a watch strap.

As discussed earlier, the PPG sensors, which include LEDs and photodetectors, are located on the side of the back surface of the wearable device. Thus, during operation, the radiation surface of the PPG sensors is in contact with the user's hand wrist. Several PPG sensors may be provided for in the wearable device 201 to provide at least three radiation sources and at least two radiation detectors.

In an embodiment of one or more embodiments of the present disclosure shown in FIG. 9B, the wearable device 201 comprises five radiation sources 221 and four radiation detectors 222, wherein these radiation sources 221 emit a light of different wavelengths in the range of 500-1000 nm. In an embodiment of one or more embodiments of the present disclosure, each of the radiation sources 221 is a LED and each of the radiation detectors 222 is a photodetector. Light barriers may be provided between the radiation sources and/or the radiation detectors to reduce noises and eliminate crosstalks.

In addition, the wearable device 201 may have four electrodes of the BIA sensor, two sensors 251, 253 of which are integrated into control buttons of the device, and the other two sensors 252, 254 are located on the back side 215 of the wearable device.

The input device 209 is configured to receive data that may be used by another component (for example, the processor 203) of the wearable device 201, input from outside (for example, by a user of the wearable device 201). In one of one or more embodiments of the present disclosure, the input device 209 may be configured to input a user profile data. The processor 203 of the wearable device 201 may receive the user profile data from the input device 209, store in or read it from the memory 212, and use it, if necessary. Data voice input and output may also be provided for in the wearable device 201.

The user profile data may include gender, age, height, weight of the user, values of blood parameters, including glycated hemoglobin, for previous periods. The output device 210 may be configured to output an information to the user on the first side 214 of the wearable device 201. The output device 210 may include a display (module) 211, which may be configured to display an output information and is located on the first side 214 of the wearable device 201. Further, the display 211 may be configured to use an on-screen keyboard to input data such as, for example, a user profile.

The memory 212 in a preferred embodiment may be configured to store a user profile data, sets of measurement data, date and time of collecting of each measurement data set, glycated hemoglobin values determined for each measurement data set, etc.

The memory 212 may also store various instructions that, when executed on the processor 203, cause the processor 203 to control the components of the wearable device 201 associated with the processor 203 and to perform various data processing or calculations.

The above data, which the memory 212 may store, may also be stored on a remote server and/or in a cloud storage. The communication module 213 may be used to transmit data from or to the wearable device. According to one or more embodiments, the communication module 213 may include a wireless communication module 223 (for example, a cellular/mobile communication module, a short-range wireless communication module, or a global navigation satellite system (GNSS) communication module) or a wired communication module 224 (for example, a local area network (LAN) communication module, or a power line communication (PLC) module). Thus, the communication module 213 can support establishing a wireless communication channel between the wearable device 201 and an external device (for example, the device 225, the device 226, the server 227 or the cloud storage 228) and performing communication via the established communication channel. The communication module 213 may include one or more communication processors that operate independently of the processor 203 and support direct (for example, wired) communication or wireless communication. The respective one of these communication modules may communicate with an external electronic device via a first network 228 (for example, a short-range communication network such as Bluetooth™, wireless communication (Wi-Fi), or infrared data transmission (IrDA)) or a second network 229 (for example, a long-range communication network such as a cellular/mobile network, the Internet, or a computer network, for example, a local area network (LAN) or a wide area network (WAN)). These different types of communication modules may be implemented as a single component (for example, a single chip) or may be multi-component (for example, a plurality of chips).

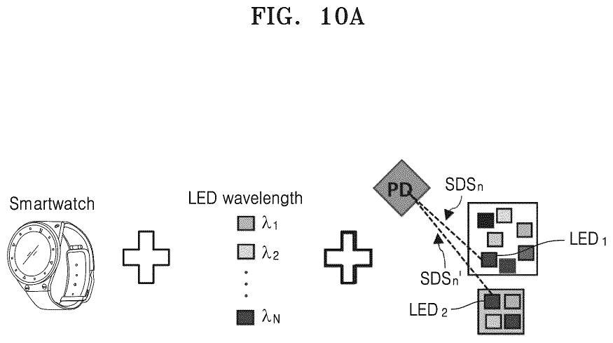

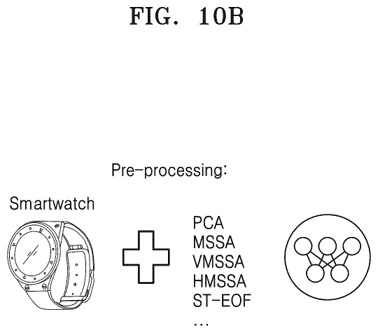

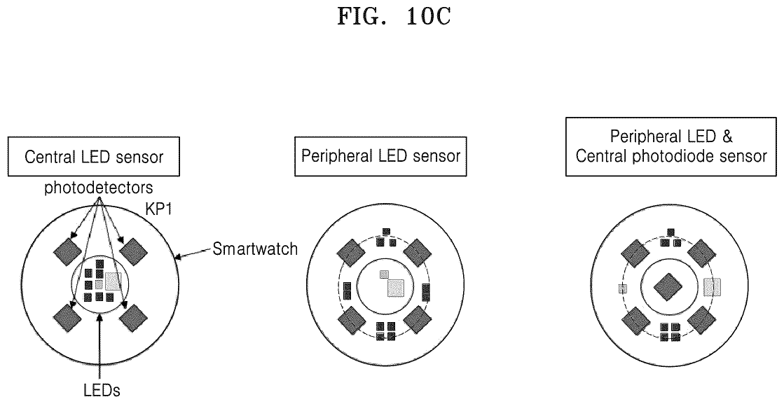

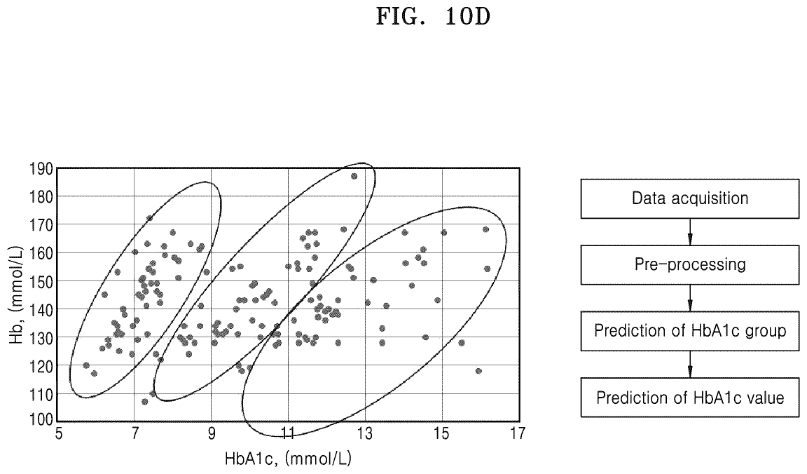

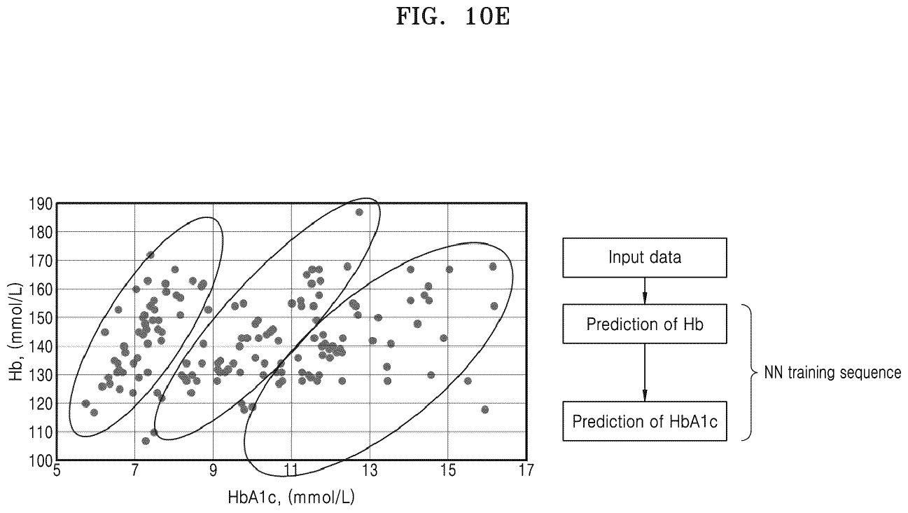

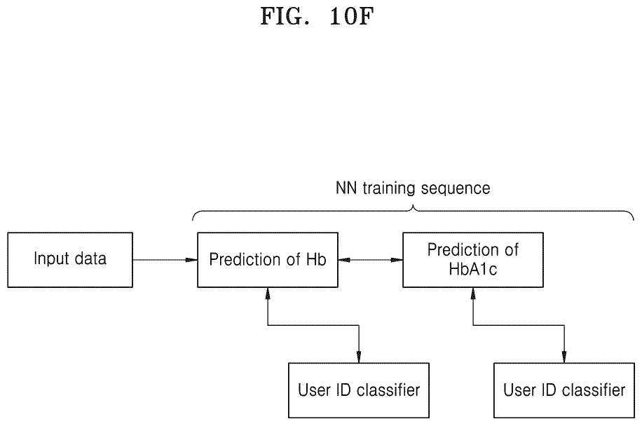

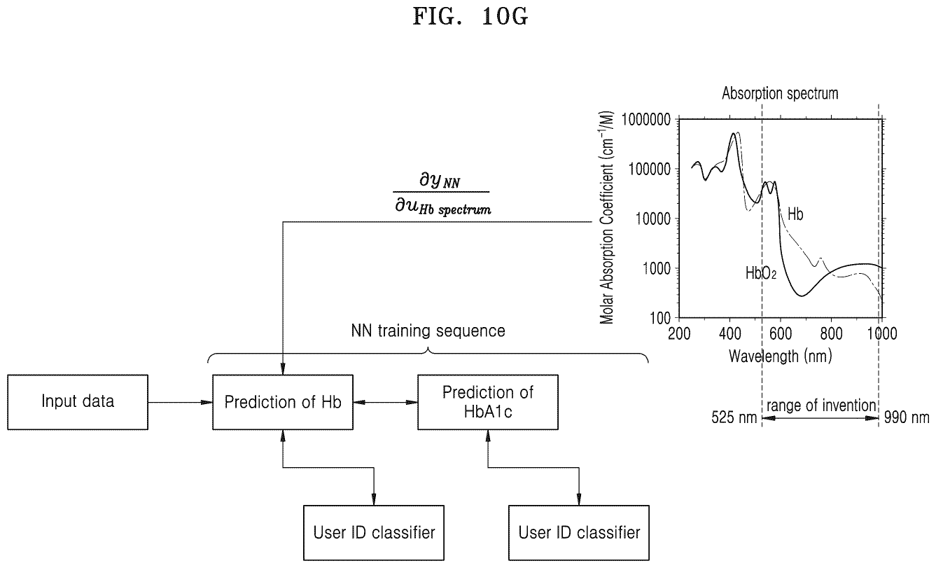

FIGS. 10A, 10B, 10C, 10D, 10E, 10F, 10G illustrate embodiments of one or more embodiments of the present disclosure.

According to an embodiment illustrated in FIG. 10A, a smartwatch uses the additional number of LEDs (e.g. including LED1 and LED2), which are located at different distances from photodetectors (e.g. PD) and emit radiation of the same and/or different wavelengths.

According to an embodiment illustrated in FIG. 10B, various methods of pre-processing of the acquired signals, such as PCA, MSSA, VMSSA, HMSSA, ST-EOF are used in the smartwatch.

These embodiments provide accurate measurement of a glycated hemoglobin level.

An embodiment illustrated in FIG. 10C is distinguished by a different positional relationship of radiation sources and detectors in the smartwatch. In particular, in a “sensor with LEDs in the center” layout, which is illustrated in the left part of FIG. 10C, all the LEDs are located in the central portion of the back side of the smartwatch, that is, within an imaginary circle inscribed between the four photodetectors, which are located along circumference at the back side of the smartwatch at intervals of 90°. In a “sensor with LEDs at the periphery” layout, which is illustrated in the central part of FIG. 10C, part of the LEDs are within said imaginary circle, and the other, most part of the LEDs are on or adjacent to the same circle on which the photodetectors are located. In a “sensor with LEDs at the periphery and central photodetector” layout, which is illustrated in the right part of FIG. 10C and is similar to the “sensor with LEDs at the periphery” layout, an additional photodetector is in the central portion instead of the LEDs.

The embodiment with the different sensor layouts provides the effect consisting in improving the determination of a glycated hemoglobin level, and also increases the diversity of specific signal features for further processing.

According to an embodiment illustrated in FIG. 10D, the prediction of HbA1c is performed in the following sequence: 1) first, it is predicted which one of the three groups (marked by ovals on graph) a glycated hemoglobin level to be determined falls into; 2) then a specific HbA1c level is predicted within the previously predicted group. Meanwhile, sequence of steps of the method for determining a glycated hemoglobin level can be represented as follows: data acquiring-pre-processing-predicting of a HbA1c group-predicting of a HbA1c value.

According to an embodiment illustrated in FIG. 10E, a linear trend between a total hemoglobin (tHb) level and a glycated hemoglobin (εHbA1c) level is observed within each group (marked by ovals in graph) (concentrations are present in mmol/L). Therefore, the prediction of HbA1c is performed in the following sequence: 1) first, a total hemoglobin (tHb) value is predicted (which is much easier to do due to the much higher concentration); 2) a glycated hemoglobin (εHbA1c) value is then predicted. Meanwhile, training of the machine learning model is performed in the sequence of “predicting of tHb-predicting of HbA1c.” The linear trend within each group provides improvement of efficiency of predicting a HbA1c level in case of improvement of efficiency of predicting tHb.

An embodiment illustrated in FIG. 10F includes using a user ID classifier during training process of the machine learning model to take into account individual characteristics of a body.

An embodiment illustrated in FIG. 10G includes using a Physics Informed Neural Network (PINN) approach during training the machine learning model. In other words, differentiation in relation to a total hemoglobin (tHb) spectrum or any other physiological/biological feature is performed. The right part of FIG. 10G show the molar absorption coefficients of tHb and HbO₂ as a function of the radiation wavelength in the range of 500-1000 nm used in the present disclosure.

The graphs are given according to a publications by Hossain S., Satter S., Kwon T.-H., Kim K.-D., Optical Measurement of Molar Absorption Coefficient of HbA1c: Comparison of Theoretical and Experimental Results, Sensors 2022, 22, 8179, https://doi.org/10.3390/s22218179/4/and Hossain S., Kwon T.-H., Kim K.-D., Estimation of molar absorption coefficients of HbA1c in near UV-Vis-SW NIR light spectrum, JKICS 2021, 46, 1030-1039/5/. Since diabetes is a group of serious diseases that can cause a plurality of complications, including sudden death, a reliable diabetes diagnostic tool, for example, glycated hemoglobin testing is needed. In order to optically estimate an amount of HbA1c present in erythrocytes, it is necessary to know the molar absorption coefficient (absorptance) of HbA1c. In said publications, the molar absorption coefficient of HbA1c was calculated on the basis of the transmission spectrum of glycated hemoglobin dissolved in water, and values of a range were calibrated with the molar absorption coefficients of glycated hemoglobin at 535 nm (green light) and 593 nm (yellow light). The molar absorption coefficient in/4/was estimated in the range of 300 to 1100 nm and a characteristic HbA1c peak at 411 nm and also two peaks at 540 nm and 576 nm were obtained. The characteristic and secondary peaks described in the publications/4/,/5/and Sanghamitra Mandal and M. O. Manasreh, An In-Vitro Optical Sensor Designed to Estimate Glycated Hemoglobin Levels, Sensors 2018, 18(4), 1084; https://doi.org/10.3390/s18041084/6/, have been confirmed by other studies, and the calculated molar absorption coefficient in the range from near UV to short-wave near-IR radiation contributes to the possibility of non-invasively determining of HbA1c.

It is clearly seen from the graph of FIG. 10G that in the range of 500-600 nm, the molar absorption coefficients of oxyhemoglobin (HbO₂) and total hemoglobin (tHb) are very similar. In addition, it is known that the absorption bands of visible light-near IR radiation (VIS-NIR) for HbO₂ and glycated hemoglobin (εHbA1c) often coincide. Hence, there is a strong mutual influence of the absorption spectra of oxygenated and glycated hemoglobins. Therefore, separate identifiers for HbO₂ and HbA1c are needed, they should be measured in parallel and decoupled during processing, and the machine learning model for estimating a glycated hemoglobin level should have multi-output prediction that takes into account glycated hemoglobin as well as at least oxygenated hemoglobin and total hemoglobin.

One or more embodiments of the disclosure make it possible to fully take into account the above problems and take into account the influence of tHb and HbO₂ on the results of determining of glycated hemoglobin.

The present disclosure provides the following advantages of the claimed solution including the method for measuring a HbA1c level:
1. The method is non-invasive/no skin damage.
2. The method is resistant to noise when predicting a HbA1c level.
3. No calibration required.

At the same time, the following effects are provided:
1. The solution is low cost, compact, and easy to use.
2. The possibility of integrating into a wearable device.
3. Obtaining a reliable information about HbA1c level status.
4. High accuracy of HbA1c level determination.

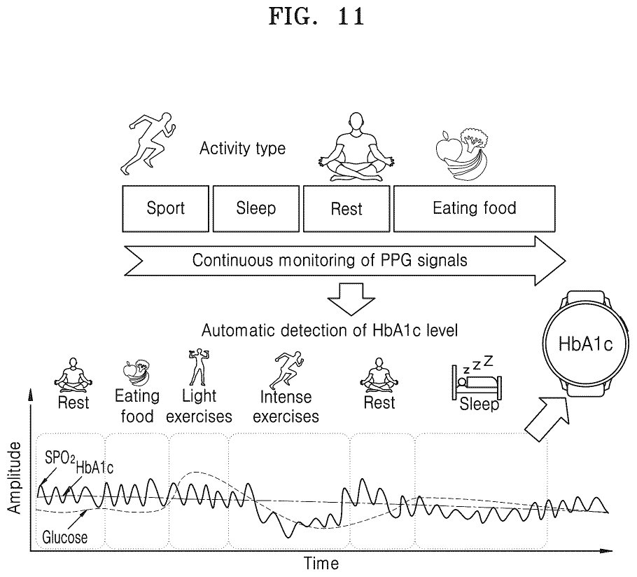

FIG. 11 illustrates occasional events in the daily life of a user of the wearable device according to one or more embodiments of the disclosure and their influence on changing of blood parameters. The daily life of a user of the wearable device, just as any person, consists of periods of sleep, rest, eating, reading, sport activities, etc., that is, some physiological events occur from time to time. As known, with almost any event that occurs in a person's daily life, changes in values of some blood components occur, for example, after eating, the glucose level rises, during intense physical activity, the glucose level, on the contrary, falls and the blood oxygenation sharply decreases. The blood oxygenation also decreases during sleep. Thus, during activity periods, some blood parameters change, whereas the glycated hemoglobin level practically does not change. Since the wearable device is able to provide continuous or on-demand monitoring of PPG signals during the normal life of a user, it is capable of performing an automatic estimation of a glycated hemoglobin level while taking into account the influence of other blood components.

Thus, a user of the wearable device has the possibility of automatic determination of a HbA1c level without using of additional means. Meanwhile, if a user wears the device for a long period of time, the wearable device further provides automatic detecting of the user activity by means of inertial movement sensors, gyroscopes, accelerometers and the PPG sensor unit. For example, as shown in FIG. 11, the user activity type, for example, periods of sport activities, sleep, rest, eating, etc. can be determined. This contributes to the determination of a glycated hemoglobin level, since almost any activity of the user results to changing in some blood parameters, for example, a HbO₂ level and a blood glucose level, whereas a HbA1c level is a long-term indicator of blood glucose over the last 1-3 months, i.e. it changes over a much longer period of time and is not affected by short-term changes in a blood glucose level caused by eating, exercising, etc. on a day of testing. This makes it possible to more accurately differentiate various blood components, and therefore to more accurately determine a glycated hemoglobin level.

It should also be noted that the pre-trained prediction model is provided along with the wearable device software, so the device does not require preliminary calibration. Meanwhile, a user can enter his/her personal data such as height, weight, age, gender, etc., into the device's memory, which may be used to more accurately predict a HbA1c level. Hence, for a new user of the wearable device according to one or more embodiments of the disclosure, the possibility of accurately measuring a glycated hemoglobin level is provided without any preliminary preparation.

Scenarios for use of the claimed device include a solution for monitoring an average glucose indicator by using of HbA1c level monitoring.

### Business Impact and Advantages of Use

By means of the wearable device disclosed herein, the following advantages and benefits from its use are provided:
monitoring an average glucose indicator over a certain period (2-3 months) by means of HbA1c monitoring;
control of development and phase of diabetes-related microvascular complications;
control of nutrition or other habits;
no action is required from side of a user;
possibility of non-professional use;
no additional hardware is required; and
providing new data for the S-Health application.

### INDUSTRIAL APPLICABILITY

As the number of people suffering from diabetes mellitus is increasing in world, and this disease can affect men, women and children of all ages in all countries, a HbA1c level is to be strictly monitored. In particular, the following target groups can be identified for which HbA1c monitoring is recommended:
children with diabetes;
diabetic patients with abnormal renal glucose threshold;
patients with type I diabetes mellitus, insulin-dependent;
pregnant women with type II diabetes;
people over 45 years of age;
people with a body mass index of more than 25;
people with a familiar risk of disease;
healthy people for the purpose of preventive control; and
other people when food ration or other habits are altered.

Also, people are at a risk group who have:
a high blood pressure;
vision problems;
disease of the cardiovascular system and/or stroke incident; and
kidney disease.

Thus, the present disclosure helps to control the disease course or predisposition to diabetes, provides the ability to measure an average glucose indicator (index) over a certain period (2-3 months), helps to improve control of the disease in order to prevent further development of diabetes. In particular, people who regularly exercise can reduce the risk of diabetes in half, and people who have lost at least 5% of their excess body weight can significantly reduce the risk of diabetes development.

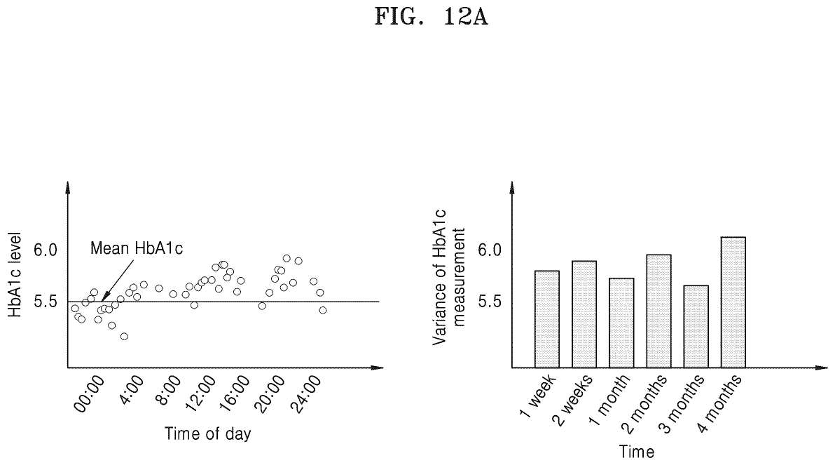

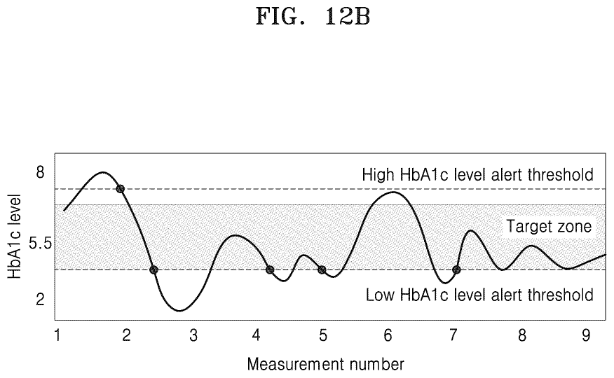

FIG. 12A schematically illustrates options of exemplary depiction of 24-hour HbA1c level measurement results and variances (deviations) of multi-month HbA1c measurement results on a screen of the wearable device according to the disclosure in the S-Health app. As illustrated in FIG. 12A, a user of the wearable device can see, on the screen of the wearable device, various data with the HbA1c measurement results over different periods of time, including, for example, specific results of single measurements and average values for a day, for a month, for a year, etc. FIG. 12B schematically illustrates a graph displayed on the screen of the wearable device in the S-Health app, which allows the variance of the glycated hemoglobin level from the target value to be monitored. With long-term HbA1c level monitoring, for example, over up to several months, variance from the target zone of the normal glycated hemoglobin level, and also crossing of high and low HbA1c level alert thresholds can be monitored. Any variance of said level from the target zone is important to be monitored. Meanwhile, when a glycated hemoglobin level is monitored, the following situations are possible. When a high or low HbA1c level going outside the target zone is determined, a user of the device is informed about the result obtained and about the preliminary diagnosis, for example, state of predisposition to diabetes, and general recommendations can be displayed on the screen: altering in diet, increase in activity, the need of outdoor walks or a recommendation to visit a doctor.

Meanwhile, short-term variances in the glycated hemoglobin level from the norm are not critical and can be corrected by a user of the device by means of recommendations displayed on the display screen. In contrast, long-term variances in the glycated hemoglobin level from the norm can mean serious health problems, and recommendations for obligatory visit to a doctor are displayed on the display screen. In addition, the data of the obtained glycated hemoglobin level monitoring results can be sent to a doctor directly from the measurment device.

The advantages of such monitoring are relatively low testing expenses, provision of a long-term period of continuous tracking of blood parameters, that provides acquiring of reliable measurement data and establishing of more accurate diagnosis of a person based on the acquired data.

The solution to the problem of estimating a glycated hemoglobin level based on the features of PPG signals relative to wavelengths and lengths of measurement channels is based on identification of empirical patterns in training data by machine learning technique.

In an embodiment of one or more embodiments of the present disclosure, the prediction model is built up for the wearable device located on a wrist because training of the prediction model was based on data recorded by sensors of the wearable device located on the wrist. In an embodiments of one or more embodiments of the present disclosure, the prediction model is possible to be trained taking into account the location of the wearable device on other parts of a body, for example, on a finger of hand. The prediction model described herein can be implemented as software including one or more instructions that can be executed by the processor 203 of the wearable device 201 or by external processors/processing devices.

In the above-described process of training the prediction model, data from profiles of a plurality of users (tested subjects), for example, gender, height, weight, age, etc., may be further used.

In addition, the prediction model can be continuously updated with new data and refined by machine learning.

In the process of machine learning, methods for selecting and processing of characteristics and corresponding discrete frequencies can be used:
in the Relief-F method, a vector of weights of features (characteristics) is calculated and normalized, and then the features whose weight exceeds a value of a predetermined threshold are selected;
the Correlation-based Feature Selection (CFS) method combines an evaluation formula with an appropriate correlation measure and a heuristic search strategy;
the Fast Correlation-based Filter method starts to operate with a whole set of features, uses a measure of symmetric uncertainty to determine dependencies between features and allows for selecting a subset by searching for and sequentially eliminating little informative features;
the Sequential Forward Feature Selection (SFFS) method at each iteration adds a feature to a set that provides the best recognition efficiency for this iteration;
the Mutual Information method determines a nonlinear correlation relationship instead of calculating the Pearson correlation “feature-feature” and “feature-tag”;
basic machine learning (artificial intelligence) algorithms; and
a combination of said regression methods and any derived regression methods, which are based on a basic algorithm, can be used as a working solution:
Decision Trees/Random Forests;
a Support Vector Machines method;
Linear Analysis;
Deep Learning methods-Artificial Neural Networks.

The advantageous effects of using the machine learning method are the ability to determine a glycated hemoglobin level for a user whose data was not used to train the prediction model, and the improved accuracy of determining a glycated hemoglobin level due to taking into account the influence of other blood components on the measurement results.

Once a prediction model is built, it can be used to estimate a glycated hemoglobin level. Meanwhile, according to the present disclosure, the influence of other blood components on the measurement results is taken into account when estimating of HbA1c. As a result, a more accurate estimation of a glycated hemoglobin level is possible than in traditional prior art methods.

Summarizing the above, the present disclosure provides an accurate estimation of a glycated hemoglobin level without the need for blood sampling, i.e. in a non-invasive manner, due to the following particulars:
KP1: Multi-signal measurement data acquisition in relation to different radiation wavelengths and different measurement channels.
KP1: Simultaneous multi-signal measurement data preparation and analysis.

As noted, a blood glucose level is tracked by means of two indicators: a glycated hemoglobin (εHbA1c) level and an instant glucose level. HbA1c is a long-term glucose indicator over the last 1-3 months and is not affected by short-term changes in a blood glucose level caused by eating, exercising, etc. on the day of testing. An instant glucose level is a short-term indicator of glucose at a particular time of testing, for example, a fasting or post-meal blood sugar level, and is prone to fluctuations due to eating and stress.

Thus, the wearable device disclosed in the present application and configured to non-invasive, continuous and/or on-demand determine a glycated hemoglobin level, suitable for non-professional use, is predicted to be in demand in the market.

### Advantageous Effects of Invention

The possibility of determination of a glycated hemoglobin level expands the functionality of the S-Health app in a smartwatch, while providing additional parameters for a comprehensive analysis of the user's health status. The smartwatch can provide daily variations, current values, a continuous glycated hemoglobin estimation result for a predetermined period of time.

Continuously monitoring of a glycated hemoglobin level provides an estimate of an average glucose indicator over a certain period of time (2-3 months), allows the direct relationship between poor monitoring and the development of complications to be determined, as well as the development and phase of microvascular complications associated with diabetes to be predicted. Such possibilities are provided due to that glucose binds to hemoglobin continuously and irreversibly during the life of erythrocytes (about 120 days), and the HbA1c level is proportional to the average blood glucose level over the past 6-12 weeks.

The advantages of HbA1c monitoring also include that the present disclosure does not depend on short-term changes in diet, physical exercises, hypoglycemic means, stress. Hence, preventive control of glycemia and feedback when treating of diabetes mellitus can be provided.

The determination of a glycated hemoglobin level by means of the wearable device disclosed herein is performed in an easy-to-use manner for a user. Moreover, as noted earlier, when the wearable device is long-termly used, automatic estimation of a glycated hemoglobin level is possible to be performed in a non-invasive manner.

Thus, the technical result of one or more embodiments of the present disclosure consists in the possibility of automatic non-invasive estimation of a glycated hemoglobin level (a long-term blood glucose indicator over the past two to three months), which is not affected by short-term changes in a blood glucose level caused by eating, physical exercises, etc. on the day of testing.

A glycated hemoglobin level has diagnostic value for determining diabetes mellitus. The HbA1c level of less than 5.7% indicates that there is currently no likelihood of developing diabetes mellitus. With the glycated hemoglobin values in the range from 5.7 to 6.5%, there are no features of diabetes mellitus, but there is a predisposition to diabetes (prediabetes). The risk of overall mortality and case fatality from stroke and myocardial infarction increases. The glycated hemoglobin level of more than 6.5% indicates the need to rule out or confirm insulin-dependent (type I) or insulin-independent (type II) diabetes mellitus and indicates the need for additional studying methods.

Possible causes of the physiological increase in a blood glucose level:
sedentary lifestyle;
high-carbohydrate food ration;
drinking insufficient amount of water;
inflammatory process in a body;
intake of certain medications (neuroleptics, steroids); and
a women menstrual period.

In patients with insulin-independent diabetes mellitus, mortality from malignancies (cancer), especially of the colorectal region, increases with the elevated glucose level. The increase in the blood glucose level always indicates of the presence of a long period of hyperglycemia and an increased risk of complications in the form of retinopathy, nephropathy, polyneuropathy, micro- and macroangiopathy. A patient suffering from hyperglycemia should strive to achieve the glycated hemoglobin level of a healthy person, i.e., 5.7%. However, this is not always possible. In such cases, the goal of therapy is considered to be a reduction of the HbA1c concentration to 6.5%. If such goal is achieved, then it can be stated that the sugar disease is sufficiently well compensated, the likelihood of the consequences of hyperglycemia in the form of all kinds of complications is reduced to a minimum level.

Blood glucose testing is also performed on women suffering from diabetes mellitus when planning pregnancy. It is established that a high content of HbA1c in six months before the onset of gestation and during the first trimester is directly dependent on the likelihood of origination of various complications of pregnancy course. Strictly monitoring of a blood glucose concentration reduces the incidence of fetal malformations from 30-40% to 2%.

The low glycated hemoglobin level (less than 4%) can be detected in an insulin-producing pancreatic tumor (insulinoma). The reason for decreasing is frequent hypoglycemic conditions. The low HbA1c concentration is detected in adrenal insufficiency and some rare hereditary diseases (Gers and Forbes diseases, hereditary fructose intolerance). In addition, possible causes of the physiological decrease in a blood glucose level include:
excessive physical activities;
abstinence from food (starvation);
long-term diet; and
use of alcoholic beverages.

An approximate ratio of a blood glucose content and a glycated hemoglobin level is presented in Table 1.

TABLE 1

| HbA1c level, % | Glucose level, mmol/l |
|---|---|
| 14.0 | 19.7 |
| 13.0 | 18.1 |
| 12.0 | 16.5 |
| 11.0 | 15.0 |
| 10.0 | 13.3 |
| 9.0 | 11.8 |
| 8.0 | 10.2 |
| 7.0 | 8.6 |
| 6.0 | 7.0 |
| 5.0 | 5.4 |

The following diseases and states also affect glycated hemoglobin indicators:
acute (recent) or chronic bleeding may underestimate a real level of glycated hemoglobin;
with iron deficiency anemia, the result of the study of glycated hemoglobin may be overestimated; and
recent blood transfusions, hemolytic anemia can be the cause of underestimated indicators of glycated hemoglobin.

Thus, the possibility of accurate estimation of a glycated hemoglobin level by means of the wearable device according to the present disclosure facilitates monitoring of a user's health state.

Although the disclosure has been described with some illustrative embodiments, it should be understood that the disclosure is not limited to these specific embodiments. In contrary, the disclosure is intended to include all alternatives, modifications, and equivalents that can be included within the spirit and scope of the claims.

In addition, the disclosure includes all equivalents of the claims, even if the claims will be modified in a process of consideration.

What is claimed is:

1. A wearable device for determining a glycated hemoglobin level, the wearable device comprising:
at least three radiation sources configured to irradiate biological tissues of a user with radiation of at least three different wavelengths;
at least two radiation detectors, each of the at least two radiation detectors being configured to detect photoplethysmographic (PPG) signals representing the radiation of the at least three different wavelengths backscattered by the biological tissues; and
at least one processor configured to:
activate the at least three radiation sources and the at least two radiation detectors in pairs of measurement channels for the PPG signals;
sequentially detect the PPG signals through each of the measurement channels by switching the measurement channels during a measurement cycle of the PPG signals;
form, from the detected PPG signals, a dataset including a time series of values of the PPG signals relative to the at least three different wavelengths and relative to lengths of the measurement channels, and which defines an absorption of the PPG signals by the biological tissues of the user;
generate a pre-processed dataset by carrying out a pre-processing of the dataset; and
determine a glycated hemoglobin level based on the pre-processed dataset by a pre-trained machine learning model.

2. The wearable device according to claim 1, wherein each measurement channel comprises a switching frequency, in a range of 25 to 100 Hz, and a measurement cycle of at least 60 seconds.

3. The wearable device according to claim 1, wherein the pre-processing of the dataset comprises filtering artifacts from the signals and frequency filtering.

4. The wearable device according to claim 1, further comprising:
a housing;
a battery;
a memory;
an input and output device; and
a communication module accommodated in the housing,
wherein the memory is configured to store a database with results of determining the glycated hemoglobin level,
wherein the input and output device is configured to input additional data and to display the glycated hemoglobin level, and
wherein the communication module is configured to communicate with any of a remote server and a cloud storage.

5. The wearable device according to claim 1, wherein the at least three different wavelengths are in a visible (VIS) to near-infrared (NIR) spectral range.

6. The wearable device according to claim 5, wherein two wavelengths of the at least three different wavelengths are 525 nm and 575 nm.

7. The wearable device according to claim 1, wherein the dataset is an array of time series, each time series representing a plurality of sequential measurements, point by point, during the measurement cycle, and
wherein a single measurement in each of the measurement channels is represented by one of the following equations:
$$\begin{matrix}\begin{matrix}{A}_{1} = ( {ε}_{HbA 1 c} ( {λ}_{1} ) \times {c}_{HbA 1 c} + {ε}_{tHb} ( {λ}_{1} ) \times {c}_{tHb} + {ε}_{HHb} ( {λ}_{1} ) \times {c}_{HHb} + \\ … + {ε}_{absorber 1} ( {λ}_{1} ) \times {c}_{absorber 1} + {ε}_{absorber 2} ( {λ}_{1} ) \times {c}_{absorber 2} + … ) \times {d}_{1} ,\end{matrix} \\ \begin{matrix}{A}_{2} = ( {ε}_{HbA 1 c} ( {λ}_{1} ) \times {c}_{HbA 1 c} + {ε}_{tHb} ( {λ}_{1} ) \times {c}_{tHb} + {ε}_{HHb} ( {λ}_{1} ) \times {c}_{HHb} + \\ … + {ε}_{absorber 1} ( {λ}_{1} ) \times {c}_{absorber 1} + {ε}_{absorber 2} ( {λ}_{1} ) \times {c}_{absorber 2} + … ) \times {d}_{N} ,\end{matrix} \\ … \\ \begin{matrix}{A}_{N} = ( {ε}_{HbA 1 c} ( {λ}_{n} ) \times {c}_{HbA 1 c} + {ε}_{tHb} ( {λ}_{n} ) \times {c}_{tHb} + {ε}_{HHb} ( {λ}_{n} ) \times {c}_{HHb} + \\ … + {ε}_{absorber 1} ( {λ}_{n} ) \times {c}_{absorber 1} + {ε}_{absorber 2} ( {λ}_{n} ) \times {c}_{absorber 2} + … ) \times {d}_{N} ,\end{matrix}\end{matrix}$$
where A₁ . . . A_{N} is a total absorption in the measurement channels 1 . . . N, respectively,
λ₁ . . . λₙ is a wavelength of radiation,
d₁ . . . d_{N} is a length of a measurement channel,
ε is a molar absorption coefficient of a particular absorber, and
c is a concentration of the particular absorber.

8. The wearable device according to claim 7, wherein the equations further comprise additional parameters representing at least one of an ambient temperature, a heart rate, and body movements.

9. The wearable device according to claim 7, wherein the equations are based on an isobestic point at a wavelength of 730 nm, 805 nm, or 850 nm.

10. The wearable device according to claim 7, wherein the equations further comprise parameters representing an influence of dark noise and individual characteristics of a body.

11. The wearable device according to claim 1, wherein the machine learning model is pre-trained by using a plurality of datasets acquired during a measurement cycle of at least 300 seconds and correlated with reference glycated hemoglobin level data measured by a laboratory technique.

12. The wearable device according to claim 11, wherein the machine learning model is trained by using different measurement channels, different radiation wavelengths, and reflects influence of additional parameters, dark noise, individual characteristics of a body, and an isobestic point.

13. The wearable device according to claim 1, wherein the wearable device is configured to be worn on a wrist.

14. A method for determining a glycated hemoglobin level, performed by a wearable device, the method comprising:
irradiating, by at least three radiation sources, biological tissues of a user with radiation of at least three different wavelengths,
detecting, by at least two radiation detectors, photoplethysmographic (PPG) signals representing the radiation of the at least three different wavelengths backscattered by the biological tissues,
wherein the detecting the PPG signals comprises:
forming measurement channels for the PPG signals by activating pairs of the at least three radiation sources and the at least two radiation detectors;
sequentially detecting the PPG signals by switching the measurement channels during a measurement cycle;
forming, from the detected PPG signals, a dataset which includes a time series of values of the PPG signals relative to the at least three different wavelengths and relative to lengths of the measurement channels, and which defines an absorption of the PPG signals by the biological tissues of the user;
generating a pre-processed dataset by pre-processing the dataset; and
determining a glycated hemoglobin level based on the pre-processed dataset by a pre-trained machine learning model.

15. The method according to claim 14, wherein each measurement channel comprises a switching frequency, in a range of 25 to 100 Hz, and a measurement cycle of at least 60 seconds.

16. The method according to claim 14, wherein the pre-processing of the dataset comprises filtering artifacts from the signals and frequency filtering.

17. The method according to claim 14, wherein the at least three different wavelengths are in a visible (VIS) to near-infrared (NIR) spectral range.

18. The method according to claim 14, wherein the dataset is an array of time series, each time series representing a plurality of sequential measurements, point by point, during the measurement cycle, and
wherein a single measurement in each of the measurement channels is represented by one of the following equations:
$$\begin{matrix}\begin{matrix}{A}_{1} = ( {ε}_{HbA 1 c} ( {λ}_{1} ) \times {c}_{HbA 1 c} + {ε}_{tHb} ( {λ}_{1} ) \times {c}_{tHb} + {ε}_{HHb} ( {λ}_{1} ) \times {c}_{HHb} + \\ … + {ε}_{absorber 1} ( {λ}_{1} ) \times {c}_{absorber 1} + {ε}_{absorber 2} ( {λ}_{1} ) \times {c}_{absorber 2} + … ) \times {d}_{1} ,\end{matrix} \\ \begin{matrix}{A}_{2} = ( {ε}_{HbA 1 c} ( {λ}_{1} ) \times {c}_{HbA 1 c} + {ε}_{tHb} ( {λ}_{1} ) \times {c}_{tHb} + {ε}_{HHb} ( {λ}_{1} ) \times {c}_{HHb} + \\ … + {ε}_{absorber 1} ( {λ}_{1} ) \times {c}_{absorber 1} + {ε}_{absorber 2} ( {λ}_{1} ) \times {c}_{absorber 2} + … ) \times {d}_{1} ,\end{matrix} \\ … \\ \begin{matrix}{A}_{N} = ( {ε}_{HbA 1 c} ( {λ}_{n} ) \times {c}_{HbA 1 c} + {ε}_{tHb} ( {λ}_{n} ) \times {c}_{tHb} + {ε}_{HHb} ( {λ}_{n} ) \times {c}_{HHb} + \\ … + {ε}_{absorber 1} ( {λ}_{n} ) \times {c}_{absorber 1} + {ε}_{absorber 2} ( {λ}_{n} ) \times {c}_{absorber 2} + … ) \times {d}_{N} ,\end{matrix}\end{matrix}$$
where A₁ . . . A_{N} is a total absorption in the measurement channels 1 . . . N, respectively,
λ₁ . . . λₙ is a wavelength of radiation,
d₁ . . . d_{N} is a length of a measurement channel,
ε is a molar absorption coefficient of a particular absorber, and
c is a concentration of the particular absorber.

19. The method according to claim 14, wherein the machine learning model is pre-trained using a plurality of datasets acquired during a measurement cycle of at least 300 seconds and correlated with reference glycated hemoglobin level data measured by a laboratory technique.

20. A non-transitory computer-readable storage medium, having a computer program stored thereon that performs, when executed by a processor, the method according to claim 14.
